(19) United States
(12) Patent Application Publication
(10) Pub. No.: US 2010/0173937 A1
(43) Pub. Date: Jul. 8, 2010

(54) USE OF TETRAKIS-QUATERNARY AMMONIUM SALTS AS PAIN MODULATING AGENTS

(75) Inventors: Joseph R. HOLTMAN, Lexington, KY (US); Peter A. Crooks, Nicholasville, KY (US); Linda P. Dwoskin, Lexington, KY (US); J. Michael McIntosh, Salt Lake City, UT (US)

Correspondence Address: CROWELL & MORING LLP;INTELLECTUAL PROPERTY GROUP, P.O. BOX 14300, WASHINGTON, DC, 20044-4300, US

(73) Assignees: University of Kentucky Research Foundation, Lexington, KY (US); University of Utah, Salt Lake City, UT (US)

(21) Appl. No.: 12/632,339

(22) Filed: Dec. 7, 2009

Related U.S. Application Data
(60) Provisional application No. 61/200,969, filed on Dec. 5, 2008.

(51) Int. Cl. A61K 31/472; A61K 31/03; A61K 31/44; A61P 29/00

(52) U.S. Cl. 514/308; 514/751; 514/332; 514/333

(57) ABSTRACT

Provided are methods of modulating, treating, reversing and/or preventing pain using tetrakis quaternary ammonium compounds, especially regarding pain of central and/or peripheral origin and/or pain which is nociceptive, neuropathic, somatic, visceral, and/or inflammatory in nature.

### CROSS-REFERENCE TO RELATED APPLICATIONS

This application claims priority under 35 U.S.C. §119 to U.S. Provisional Application No. 61/200,969, filed on Dec. 5, 2008, the entire content of which is hereby incorporated by reference in its entirety.

### FIELD OF THE INVENTION

Provided are methods of modulating, treating, reversing and/or preventing pain using tetrakis quaternary ammonium compounds, especially regarding pain of central and/or peripheral origin and/or pain which is nociceptive, neuropathic, somatic, visceral, and/or inflammatory in nature.

### BACKGROUND OF THE INVENTION

The treatment of pain is a critical health issue. Acute pain, such as post-operative pain, and chronic pain, such as arthritis, lower back and cancer pain, affects tens of millions of people annually in the US. Each year some 30 million people visit a physician with a complaint of a painful condition. Some 10% of these patients are seen with chronic pain as their main complaint. The financial loss due to pain has been estimated to exceed 100 billion dollars a year as a result of medical fees, decreased productivity, litigation and the cost of drugs.

Pain can be broadly divided into the categories of nociceptive, neuropathic and inflammatory pain. Nociceptive pain occurs as a result of activation of peripheral nociceptors, actually free nerve endings by noxious stimuli (heat, pressure, inflammatory mediators). Examples of nociceptive pain include postsurgical pain, inflammatory pain, (such as arthritis) and lower back pain. Neuropathic pain occurs as a result of damage to the peripheral or central nervous system. Examples of neuropathic pain include radiculopathy (such as from disc impingement on a nerve), complex regional pain syndrome (such as CRPS I and II), diabetic peripheral neuropathy or central pain (such as that from stroke, spinal cord injury, and multiple sclerosis). Patients may describe neuropathic pain as “burning and tingling” in nature, and this pain is usually characterized by hyperalgesia (increased painful response to a noxious stimulus) and allodynia (pain to a previously non-noxious stimulus). Inflammatory pain is often localized, and often caused by an insult to the integrity of tissues at a cellular level. Accordingly, inflammatory pain may be associated with cellular injury caused by penetration wounds, burns, extreme cold, fractures, arthritis, autoimmune conditions, excessive stretching, infections and vasoconstriction.

In many pain patients, in particular those with chronic pain conditions of both malignant, cancer-related pain and non-malignant origin, pain is often inadequately managed with currently available drugs. Available drugs are often merely simple modifications of drugs from classes which have been available for decades including the opioids, nonsteroidal anti-inflammatory agents (NSAID's) or various adjuvants, such as antidepressants and anticonvulsants, initially approved for other uses besides pain. Opioids are often successfully used for the treatment of moderate to severe nociceptive pain. However, chronic neuropathic pain is much less responsive to opioids. Use of opioid analgesics is associated with a broad range of significant side effects including cognitive impairment, respiratory depression and constipation. In addition, long-term opioid dosing results in the development of tolerance to the analgesic effect, drug abuse and dependence. The NSAID's (such as ibuprofen) act by inhibition of the cyclo-oxygenase (COX) enzyme. They are especially useful in nociceptive pain of inflammatory origin, such as pain from arthritis. However, the NSAID's have limited efficacy when compared to the opioids. In addition, NSAID's have significant side effects, including potential damage to the renal, gastrointestinal, and cardiovascular systems.

The discovery of COX-2 selective agents, including rofecoxib (Vioxx®), celecoxib (Celebrex®), valdecoxib (Bextra®), which have far less gastrointestinal toxicity, was thought to be an advance in NSA/D pharmacology. Nonetheless, these agents still have low efficacy and evidence is now available linking them to significant cardiovascular events including stroke and myocardial infarction following chronic use, resulting in the removal of rofecoxib and valdecoxib from the market. Accordingly, no truly efficacious agent exists for the treatment of neuropathic pain.

GABA-pentin (Neurontin®), an anticonvulsant, has found use for some neuropathic pain syndromes, but it still has limited efficacy. Duloxetine (Cymbalta®), an antidepressant, has recently been approved for the treatment of diabetic peripheral neuropathy. However, it has limited efficacy and usefulness for other neuropathic pain states. The N-methyl-d-aspartate (NMDA) receptor antagonists (such as ketamine) have been proposed for the treatment of neuropathic pain. Their general use is impractical given the marked side effects including sedation, psychosis and motor impairment. The limitations of the currently available therapies clearly demonstrate the need for a broad spectrum new class of efficacious and safe analgesic drugs for the treatment of nociceptive and neuropathic pain.

New therapeutic agents with broader efficacy, for nociceptive, neuropathic and mixed nociceptive-neuropathic pain syndromes, and with fewer side effects would result in significant societal benefit. Given the need for more effective, less toxic, analgesic drugs, a great deal of emphasis has been placed on identifying novel molecular targets that could form the basis for new analgesics. One of the promising new targets is the neuronal nicotinic acetylcholine receptor (nAChR). nAChR's play an important role in the control of pain and thus drugs acting at the nicotinic receptor can be expected to have analgesic properties. The tetrakis-quaternary ammonia salts are able to interact with the nACHR.

Nicotinic receptor drugs have a broad spectrum of analgesic activity in several preclinical animal models of pain of nocicpetive and neuropathic origin. This includes acute thermal pain models (as shown in tail flick and hot plate testing), inflammatory pain models (formalin or carrageenan injection into the paw) and nerve injury models (spinal or sciatic nerve ligation). Both anti-hyperalgesic and anti-allodynic effects were observed in the neuropathic pain models.

Thus, it appears that nicotinic drugs have promise as analgesic agents for the treatment of several types of clinical pain including nociceptive, neuropathic and inflammatory pain.

### SUMMARY OF INVENTION

Compounds contemplated for use in the present invention may include the following:

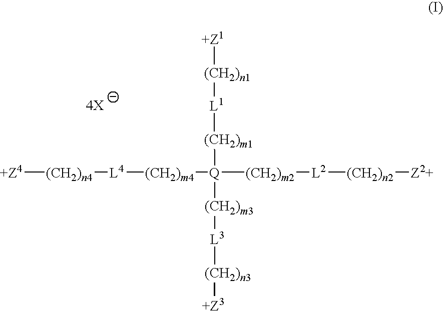

Each X^{θ} is independently an organic or inorganic anion.

Q is a phenyl group substituted at the 1-, 2-, 3- and 4-positions, at the 1-, 2-, 3- and 5-positions, or at the 1-, 2-, 4- and 5-positions.

The values for m1, m2, m3 and m4 are each independently 0, 1, 2, 3, 4 or 5.

The values for n1, n2, n3 and n4 are each independently 1, 2, 3, 4 or 5.

L¹, L², L³ and L⁴ are each independently selected from —CH₂CH₂—, cis —CH═CH—, trans —CH═CH—, —C≡C—, —S—CH₂—, —CH₂—S—, —Se—CH₂—, —CH₂—Se—, —O—CH₂—, —CH₂—O—, —NH—CH₂—, —CH₂—NH—, —N(lower alkyl)-CH₂—, —CH₂—N(lower alkyl)-, —N═CH—, —CH═N— or —N═—.

Z¹, Z², Z³ and Z⁴ are each independently five or six membered rings as shown in formulas (IIA) and (IIB), wherein each ring of Z¹, Z², Z³ and Z⁴ has one, two or three nitrogen atoms.

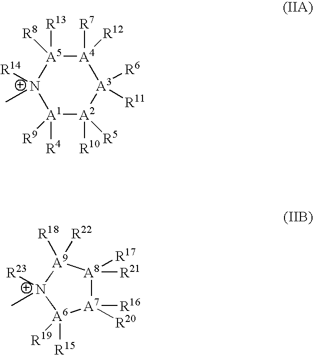

A¹ is carbon or nitrogen, provided that when A¹ joins a ring atom with an unsaturated bond or is a nitrogen, R⁹ is absent, and when A¹ joins a ring atom with an unsaturated bond and is a nitrogen, both R⁴ and R⁹ are absent. A² is carbon or nitrogen, provided that when A² joins a ring atom with an unsaturated bond or is a nitrogen, R¹⁰ is absent, and when A² joins a ring atom with an unsaturated bond and is a nitrogen, both R⁵ and R¹⁰ are absent. A³ is carbon or nitrogen, provided that when A³ joins a ring atom with an unsaturated bond or is a nitrogen, R¹¹ is absent, and when A³ joins a ring atom with an unsaturated bond and is a nitrogen, both R⁶ and R¹¹ are absent. A⁴ is carbon or nitrogen, provided that when A⁴ joins a ring atom with an unsaturated bond or is a nitrogen, R¹² is absent, and when A⁴ joins a ring atom with an unsaturated bond and is a nitrogen, both R⁷ and R¹² are absent. A⁵ is carbon or nitrogen, provided that when A⁵ joins a ring atom with an unsaturated bond or is a nitrogen, R¹³ is absent, and when A⁵ joins a ring atom with an unsaturated bond and is a nitrogen, both R⁸ and R¹³ are absent. A⁶ is carbon or nitrogen, provided that when A⁶ joins a ring atom with an unsaturated bond or is a nitrogen, R¹⁹ is absent, and when A⁶ joins a ring atom with an unsaturated bond and is a nitrogen, both R¹⁵ and R¹⁹ are absent. A⁷ is carbon or nitrogen, provided that when A⁷ joins a ring atom with an unsaturated bond or is a nitrogen, R²⁰ is absent, and when A⁷ joins a ring atom with an unsaturated bond and is a nitrogen, both R¹⁶ and R²⁰ are absent. A⁸ is carbon or nitrogen, provided that when A⁸ joins a ring atom with an unsaturated bond or is a nitrogen, R²¹ is absent, and when A⁸ joins a ring atom with an unsaturated bond and is a nitrogen, both R¹⁷ and R²¹ are absent. A⁹ is carbon or nitrogen, provided that when A⁹ joins a ring atom with an unsaturated bond or is a nitrogen, R²² is absent, and when A⁹ joins a ring atom with an unsaturated bond and is a nitrogen, both R¹⁸ and R²² are absent.

R¹⁴ or R²³ is absent when any of the bonds to the ammonium nitrogen is unsaturated, and R¹⁴ or R²³ is a straight chain or branched alkyl group of four carbons or fewer when all of the bonds to the ammonium nitrogen are saturated. R⁴, R⁵, R⁶, R⁷, R⁸, R⁹, R¹⁰, R¹¹, R¹², and R¹³ or R¹⁵, R¹⁶, R¹⁷, R¹⁸, R¹⁹, R²⁰, R²¹, and R²², when present, are each independently selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, substituted heterocyclic, halo, cyano, nitro, SOY¹, SO₂Y¹, SO₂OY¹ or SO₂NHY¹, where Y¹ is selected from hydrogen, lower alkyl, alkenyl, alkynyl or aryl, and where Y¹ is not hydrogen in SOY¹ and if Y¹ is alkenyl or alkynyl, the site of unsaturation is not conjugated with a heteroatom; COY², where Y² is selected from hydrogen, alkyl, substituted alkyl, cycloalkyl, substituted cycloalkyl, alkoxy, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, or substituted heterocyclic, and where if Y² comprises alkenyl or alkynyl, the site of unsaturation is not conjugated with the carbonyl group; OY³, where Y³ is selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, acyl, substituted acyl, alkylsulfonyl, arylsulfonyl, heterocyclic, or substituted heterocyclic, where if Y³ comprises alkenyl or alkynyl, the site of unsaturation is not conjugated with the oxygen; NY⁴Y⁵, where Y⁴ and Y⁵ are each independently selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, acyl, substituted acyl, alkylsulfonyl, arylsulfonyl, heterocyclic, or substituted heterocyclic, where if Y⁴ or Y⁵ comprises alkenyl or alkynyl, the site of unsaturation is not conjugated with the nitrogen; SY⁶, where Y⁶ is selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, or substituted heterocyclic, and where if Y⁶ comprises alkenyl or alkynyl, the site of unsaturation is not conjugated with the sulfur; or R⁴ and R⁵ together with A¹ and A², or R⁵ and R⁶ together with A² and A³, or R¹⁵ and R¹⁶ together with A⁶ and A⁷, or R¹⁶ and R¹⁷ together with A⁷ and A⁸ independently form a three to eight member cyclolkane, substituted cycloalkane, cycloalkene, substituted cycloalkene, aryl, substituted aryl, heterocycle with one to three hetero atoms in the ring, or substituted heterocycle with one to three hetero atoms in the ring.

In another embodiment, a composition is provided comprising a pharmaceutically acceptable carrier and a compound as described above. In another embodiment, a method is provided for selectively modulating nociception and pain comprising administering a therapeutically effective amount of a compound as described above to a mammalian subject in need thereof.

Other methods, features and advantages of the present invention will be or become apparent to one with skill in the art upon examination of the following detailed descriptions. It is intended that all such additional methods, features and advantages be included within this description, be within the scope of the present invention, and be protected by the accompanying claims.

### BRIEF DESCRIPTION OF THE DRAWINGS

FIG. 1 shows the time course of the analgesic effect of ZZ-204G in the rodent formalin tonic inflammatory pain model following intraperitoneal administration. Data are mean±S.E.M., n=3 rats.

FIG. 2 show the dose response of the analgesic effect of ZZ-204G in phase 1 and 2 of the rodent formalin tonic inflammatory pain model following intraperitoneal administration. Data are mean±S.E.M., n=3 rats.

FIG. 3 shows the time course of the antihyperalgesic effect of ZZ-204G in the rodent chronic constriction nerve injury model of neuropathic pain following intraperitoneal administration. Data are mean±S.E.M., n=4-8 rats.

FIG. 4 shows the dose response of the antihyperalgesic effect of ZZ-204G in the rodent chronic constriction nerve injury model of neuropathic pain following intraperitoneal administration. Data are mean±S.E.M., n=4-8 rats.

### DETAILED DESCRIPTION OF INVENTION

Before the present compositions and methods are described, it is to be understood that the invention is not limited to the particular methodologies, protocols, assays, and reagents described, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless the context clearly dictates otherwise.

Unless defined otherwise, all technical and scientific terms used herein have the same meanings as commonly understood by one of ordinary skill in the art to which this invention belongs. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The term “agonist” refers to a substance which interacts with a receptor and increases or prolongs a physiological response (i.e. activates the receptor).

The term “partial agonist” refers to a substance which interacts with and activates a receptor to a lesser degree than an agonist.

The term “antagonist” refers to a substance which interacts with and decreases the extent or duration of a physiological response of that receptor.

The terms “disorder,” “disease,” and “condition” are used inclusively and refer to any status deviating from normal.

The term “lower alkyl” refers to straight or branched chain alkyl radicals having in the range of 1 to 4 carbon atoms.

The term “alkyl” refers to straight or branched chain alkyl radicals having 1 to 19 carbon atoms, and “substituted alkyl” refers to alkyl radicals further bearing one or more substituents including, but not limited to, hydroxy, alkoxy (of a lower alkyl group), mercapto (of a lower alkyl group), aryl, heterocyclic, halogen, trifluoromethyl, cyano, nitro, amino, carboxyl, carbamate, sulfonyl, and sulfonamide.

The term “cycloalkyl” refers to cyclic ring-containing moieties containing 3 to 8 carbon atoms, and “substituted cycloalkyl” refers to cycloalkyl moieties further bearing one or more substituents as set forth above.

The term “alkenyl” refers to straight or branched chain hydrocarbyl groups having at least one carbon-carbon double bond and having 2 to 19 carbon atoms, and “substituted alkenyl” refers to alkenyl groups further bearing one or more substituents as set forth above.

The term “alkynyl” refers to straight or branched chain hydrocarbyl moieties having at least one carbon-carbon triple bond and having 2 to 19 carbon atoms, and “substituted alkynyl” refers to alkynyl moieties further bearing one or more substituents as set forth above.

The term “aryl” refers to aromatic groups having 6 to 24 carbon atoms, and “substituted aryl” refers to aryl groups further bearing one or more substituents as set forth above.

The term “alkylaryl” refers to alkyl-substituted aryl groups, and “substituted alkylaryl” refers to alkylaryl groups further bearing one or more substituents as set forth above.

The term “arylalkyl” refers to aryl-substituted alkyl groups, and “substituted arylalkyl” refers to arylalkyl groups further bearing one or more substituents as set forth above.

The term “arylalkenyl” refers to aryl-substituted alkenyl groups, and “substituted arylalkenyl” refers to arylalkenyl groups further bearing one or more substituents as set forth above.

The term “arylalkynyl” refers to aryl-substituted alkynyl groups, and “substituted arylalkynyl” refers to arylalkynyl groups further bearing one or more substituents as set forth above.

The term “heterocyclic” refers to cyclic moieties containing one or more heteroatoms as part of the ring structure and having 3 to 24 carbon atoms, and “substituted heterocyclic” refers to heterocyclic moieties further bearing one or more substituents as set forth above.

The term “acyl” refers to alkyl-carbonyl groups, and “substituted acyl” refers to acyl groups further bearing one or more substituents as set forth above.

The term “halogen” refers to fluoride, chloride, bromide or iodide groups.

It is understood that in all substituted groups defined above, polymers arrived at by defining substituents with further substituents to themselves (e.g. substituted aryl having a substituted aryl group as a substituent which is itself substituted with a substituted aryl group, etc.) are not intended for inclusion herein. In such cases, the maximum number of such substituents is three. That is to say that each of the above definitions is constrained by a limitation that, for example, substituted aryl groups are limited to -substituted aryl-(substituted aryl)-substituted aryl.

Compounds for use in the methods of the present invention include tetrakis-quaternary ammonium salts corresponding to Formula (I):

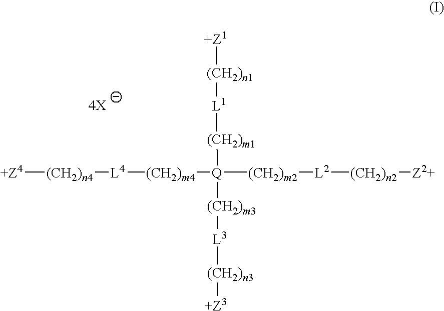

Each X^{θ} is independently an organic or inorganic anion.

Q is a phenyl group substituted at the 1-, 2-, 3- and 4-positions, at the 1-, 2-, 3- and 5-positions, or at the 1-, 2-, 4- and 5-positions.

The values for m1, m2, m3 and m4 are each independently 0, 1, 2, 3, 4 or 5.

The values for n1, n2, n3 and n4 are each independently 1, 2, 3, 4 or 5.

L¹, L², L³ and L⁴ are each independently selected from —CH₂CH₂—, cis —CH═CH—, trans —CH═CH—, —C≡C—, —S—CH₂—, —CH₂—S—, —Se—CH₂—, —CH₂—Se—, —O—CH₂—, —CH₂—O—, —NH—CH₂—, —CH₂—NH—, —N(lower alkyl)-CH₂—, —CH₂—N(lower alkyl)-, —N═CH—, —CH═N— or —N═N—.

Z¹, Z², Z³ and Z⁴ are each independently five or six membered rings as shown in formulas (IIA) and (IIB), wherein each ring of Z¹, Z², Z³ and Z⁴ has one, two or three nitrogen atoms.

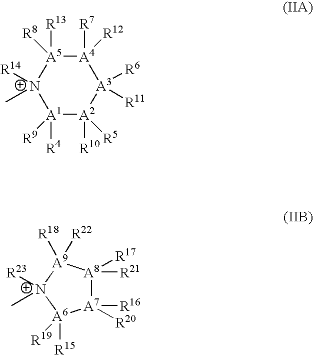

A¹ is carbon or nitrogen, provided that when A¹ joins a ring atom with an unsaturated bond or is a nitrogen, R⁹ is absent, and when A¹ joins a ring atom with an unsaturated bond and is a nitrogen, both R⁴ and R⁹ are absent.

A² is carbon or nitrogen, provided that when A² joins a ring atom with an unsaturated bond or is a nitrogen, R¹⁰ is absent, and when A² joins a ring atom with an unsaturated bond and is a nitrogen, both R⁵ and R¹⁰ are absent.

A³ is carbon or nitrogen, provided that when A³ joins a ring atom with an unsaturated bond or is a nitrogen, R¹¹ is absent, and when A³ joins a ring atom with an unsaturated bond and is a nitrogen, both R⁶ and R¹¹ are absent.

A⁴ is carbon or nitrogen, provided that when A⁴ joins a ring atom with an unsaturated bond or is a nitrogen, R¹² is absent, and when A⁴ joins a ring atom with an unsaturated bond and is a nitrogen, both R⁷ and R¹² are absent.

A⁵ is carbon or nitrogen, provided that when A⁵ joins a ring atom with an unsaturated bond or is a nitrogen, R¹³ is absent, and when A⁵ joins a ring atom with an unsaturated bond and is a nitrogen, both R⁸ and R¹³ are absent.

A⁶ is carbon or nitrogen, provided that when A⁶ joins a ring atom with an unsaturated bond or is a nitrogen, R¹⁹ is absent, and when A⁶ joins a ring atom with an unsaturated bond and is a nitrogen, both R¹⁵ and R¹⁹ are absent.

A⁷ is carbon or nitrogen, provided that when A⁷ joins a ring atom with an unsaturated bond or is a nitrogen, R²⁰ is absent, and when A⁷ joins a ring atom with an unsaturated bond and is a nitrogen, both R¹⁶ and R²⁰ are absent.

A⁸ is carbon or nitrogen, provided that when A⁸ joins a ring atom with an unsaturated bond or is a nitrogen, R²¹ is absent, and when A⁸ joins a ring atom with an unsaturated bond and is a nitrogen, both R¹⁷ and R²¹ are absent.

A⁹ is carbon or nitrogen, provided that when A⁹ joins a ring atom with an unsaturated bond or is a nitrogen, R²² is absent, and when A⁹ joins a ring atom with an unsaturated bond and is a nitrogen, both R¹⁸ and R²² are absent.

R¹⁴ or R²³ is absent when any of the bonds to the ammonium nitrogen is unsaturated, and R¹⁴ or R²³ is a straight chain or branched alkyl group of four carbons or fewer when all of the bonds to the ammonium nitrogen are saturated.

R⁴, R⁵, R⁶, R⁷, R⁸, R⁹, R¹⁰, R¹¹, R¹², and R¹³ or R¹⁵, R¹⁶, R¹⁷, R¹⁸, R¹⁹, R²⁰, R²¹, and R²², when present, are each independently selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, substituted heterocyclic, halo, cyano, nitro, SOY¹, SO₂Y¹, SO₂OY¹ or SO₂NHY¹, where Y¹ is selected from hydrogen, lower alkyl, alkenyl, alkynyl or aryl, and where Y¹ is not hydrogen in SOY¹ and if Y¹ is alkenyl or alkynyl, the site of unsaturation is not conjugated with a heteroatom; COY², where Y² is selected from hydrogen, alkyl, substituted alkyl, cycloalkyl, substituted cycloalkyl, alkoxy, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, or substituted heterocyclic, and where if Y² comprises alkenyl or alkynyl, the site of unsaturation is not conjugated with the carbonyl group; OY³, where Y³ is selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, acyl, substituted acyl, alkylsulfonyl, arylsulfonyl, heterocyclic, or substituted heterocyclic, where if Y³ comprises alkenyl or alkynyl, the site of unsaturation is not conjugated with the oxygen; NY⁴Y⁵, where Y⁴ and Y⁵ are each independently selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, acyl, substituted acyl, alkylsulfonyl, arylsulfonyl, heterocyclic, or substituted heterocyclic, where if Y⁴ or Y⁵ comprises alkenyl or alkynyl, the site of unsaturation is not conjugated with the nitrogen; SY⁶, where Y⁶ is selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, or substituted heterocyclic, and where if Y⁶ comprises alkenyl or alkynyl, the site of unsaturation is not conjugated with the sulfur; or R⁴ and R⁵ together with A¹ and A², or R⁵ and R⁶ together with A² and A³, or R¹⁵ and R¹⁶ together with A⁶ and A⁷, or R¹⁶ and R¹⁷ together with A⁷ and A⁸ independently form a three to eight member cyclolkane, substituted cycloalkane, cycloalkene, substituted cycloalkene, aryl, substituted aryl, heterocycle with one to three hetero atoms in the ring, or substituted heterocycle with one to three hetero atoms in the ring.

Z¹, Z², Z³ and Z⁴ may include pyrrole, pyrrolidine, pyrazole, imidazole, 1,2,3-triazole, 1,2,4-triazole, pyridine, piperidine, quinoline, tetrahydroquinoline, isoquinoline, tetrahydroisoquinoline, pyrazine, piperazine, pyridazine, and triazine.

R⁴, R⁵, R⁶, R⁷, R⁸, R⁹, R¹⁰, R¹¹, R¹², R¹³, R¹⁵, R¹⁶, R¹⁷, R¹⁸, R¹⁹, R²⁰, R²¹, and R²² may include hydrogen, methyl, ethyl, propyl, butyl, trifluoromethyl, hydroxymethyl, hydroxyethyl, hydroxypropyl, hydroxybutyl, phenyl, benzyl, pyrrolidine, N-alkyl pyrrolidine (for example where the alkyl chain is methyl, ethyl or propyl), unsaturated pyrrolidine, unsaturated N-alkyl pyrrolidine (for example where the alkyl chain is methyl, ethyl or propyl), aziridine, N-methyl aziridine, azetidine, N-methyl azetidine, unsaturated azetidine, unsaturated N-methyl azetidine, piperidine, N-methyl piperidine, unsaturated piperidine, unsaturated N-methyl piperidine, azepane, N-methyl azepane, unsaturated azepane, unsaturated N-methyl azepane, azocane, N-methyl azocane, unsaturated azocane, unsaturated N-methyl azocane, 1-aza-bicyclo[3.2.1]octane, 1-aza-bicyclo[2.2.1]heptane, 8-methyl-8-aza-bicyclo[3.2.1]octane, 1-aza-tricyclo[3.3.1.1^{3,7}]decane, methyl cycloalkyl, methyl substituted cycloalkyl, methylpyrrolidine, methyl N-alkyl pyrrolidine (for example where the alkyl chain is methyl, ethyl or propyl), methyl unsaturated pyrrolidine, methyl unsaturated N-alkyl pyrrolidine (for example where the alkyl chain is methyl, ethyl or propyl), methyl aziridine, methyl N-methyl aziridine; methyl azetidine, methyl N-methyl azetidine, methyl unsaturated azetidine, methyl unsaturated N-methyl azetidine, methyl piperidine, methyl N-methyl piperidine, methyl unsaturated piperidine, methyl unsaturated N-methyl piperidine, methyl azepane, methyl N-methyl azepane, methyl unsaturated azepane, methyl unsaturated N-methyl azepane, methyl azocane, methyl N-methyl azocane, methyl unsaturated azocane, methyl unsaturated N-methyl azocane, methyl-1-aza-bicyclo[3.2.1]octane, methyl-1-aza-bicyclo[2.2.1]heptane, 8-methyl-8-aza-bicyclo[3.2.1]octane, and methyl-1-aza-tricyclo[3.3.1.1^{3,7]decane.}

As a further example, when R⁴ and R⁵ together with A¹ and A², or R⁵ and R⁶ together with A² and A³, or R¹⁵ and R¹⁶ together with A⁶ and A⁷, or R¹⁶ and R¹⁷ together with A⁷ and A⁸ independently form a three to eight-membered ring, that ring may be a heterocycle containing up to three hetero atoms (for example nitrogen, oxygen or sulfur) in the ring, and further may be substituted with one or more substituents. Rings may include benzene, pyridine, pyran, indene, isoindene, benzofuran, isobenzofuran, benzo[b]thiophene, benzo[c]thiophene, indole, indolenine, isoindole, cyclopental[b]pyridine, pyrano[3,4-b]pynrole, indazole, indoxazine, benzoxazole, anthranil naphthalene, tetralin, decalin, chromene, coumarin, chroman-4-one, isocoumarin, isochromen-3-one, quinoline, isoquinoline, 5,6,7,8-tetrahydro-isoquinoline, cinnoline, quinazoline, naphthyrdine, pyrido[3,4]-pyridine, pyridol[3,2-b]pyridine, pyrido[4,3,-b]-pyridine, benzoxazine, anthracene, phenanthrene, phenalene, fluorene, carazole, xanthene, acnidine, octahydro-[1]pyridine, 1-methyloctahydro-[1]pyridine, octahydroindole, 1-methyloctahydro-indole, octahydro-cyclopenta[b]pyrrole, 1-methyloctahydro-cyclopenta[b]pyrrole, decahydroquinoline, and 1-methyldecahydroquinoline.

X^{θ}, for example, includes F⁻, Cl⁻, Br⁻, I⁻, NO₂⁻, HSO₄⁻, SO₄⁻, HPO₄⁻, PO₄²⁻, methanesulfonate, trifluoromethane sulfate, p-toluenesulfonate, benzenesulfonate, salicylate, proprionate, ascorbate, aspartate, fumarate, galactarate, maleate, citrate, glutamate, glycolate, lactate, malate, maleate, tartrate, oxalate, succinate, or similar pharmaceutically acceptable organic acid addition salts, including the pharmaceutically acceptable salts listed in the Journal of Pharmaceutical Sciences volume 66, page 2, 1977, which are hereby incorporated by reference. The above salt forms may be in some cases hydrates or solvates with alcohols and other solvents.

In a compound of Formula (I), the phenyl ring of Q may substituted at the 1-, 2-, 4- and 5-positions. A¹, A², A³, A⁴, and A⁵ may be carbon. Z¹, Z², Z³ and Z⁴ may be substituted, six-membered, aromatic rings. To this end, Z¹, Z², Z³ and Z⁴ may be substituted pyridinium rings.

R⁴ may be hydrogen. R⁵ may be hydrogen, alkyl, hydroxyalkyl, phenyl, benzyl, 1-methyl-2-pyrrolidinyl, or forms a six-membered ring with A², A³ and R⁶. To this end, R⁵ may be hydrogen, methyl, hydroxypropyl, phenyl, benzyl, 1-methyl-2-pyrrolidinyl, forms a phenyl group with A², A³ and R⁶, or forms a cyclohexyl group with A², A³ and R⁶. R⁶ may be hydrogen, alkyl, forms a phenyl group with A², A³ and R⁵, or forms a cyclohexyl group with A², A³ and R⁵. To this end, R⁶ may be hydrogen, methyl, forms a phenyl group with A², A³ and R⁵, or forms a cyclohexyl group with A², A³ and R⁵.

R⁷ may be hydrogen or alkyl. To this end, R⁷ may be hydrogen or methyl. R⁸ may be hydrogen. m1, m2, m3 and m4 may equal 0. n1, n2, n3 and n4 may equal 3. L¹, L², L³ and L⁴ may be —CH₂—CH₂— or —C≡C—. X^{θ} may be a halogen. For example, X^{θ} may be bromide.

In one embodiment, the compound of Formula (I) is defined wherein the phenyl ring of Q is 1,2,4,5-substituted; wherein m1, m2, m3 and m4=0; wherein n1, n2, n3 and n4=3; wherein L is —CH₂CH₂— or —C≡C—; wherein Z¹, Z², Z³ and Z⁴ are pyridinium rings; wherein R⁴ is hydrogen; wherein R⁵ is hydrogen, methyl, hydroxypropyl, phenyl, benzyl, 1-methyl-2-pyrrolidinyl, forms a phenyl group with A², A³ and R⁶, or forms a cyclohexyl group with A², A³ and R⁶; wherein R⁶ is hydrogen, methyl, forms a phenyl group with A², A³ and R⁵, or forms a cyclohexyl group with A², A³ and R⁵; and wherein X^{θ} is Br.

In another embodiment, the compound of Formula (I) is defined wherein the phenyl ring of Q is 1,2,4,5-substituted; wherein m1, m2, m3 and m4=0; wherein n1, n2, n3 and n4=3; wherein L is —CH₂CH₂—; wherein Z¹, Z², Z³ and Z⁴ are pyridinium rings; wherein R⁴ is hydrogen; wherein R⁵ is hydrogen, methyl, hydroxypropyl, phenyl, benzyl, 1-methyl-2-pyrrolidinyl, forms a phenyl group with A², A³ and R⁶, or forms a cyclohexyl group with A², A³ and R⁶; wherein R⁶ is hydrogen, methyl, forms a phenyl group with A², A³ and R⁵, or forms a cyclohexyl group with A², A³ and R⁵; and wherein X^{θ} is Br.

In another embodiment, the compound of Formula (I) is defined wherein the phenyl ring of Q is 1,2,4,5-substituted; wherein m1, m2, m3 and m4=0; wherein n1, n2, n3 and n4=3; wherein L is —C≡C—; wherein Z¹, Z², Z³ and Z⁴ are pyridinium rings; wherein R⁴ is hydrogen; wherein R⁵ is hydrogen, methyl, hydroxypropyl, phenyl, benzyl, 1-methyl-2-pyrrolidinyl, forms a phenyl group with A², A³ and R⁶, or forms a cyclohexyl group with A², A³ and R⁶; wherein R⁶ is hydrogen, methyl, forms a phenyl group with A², A³ and R⁵, or forms a cyclohexyl group with A², A³ and R⁵; and wherein X^{θ} is Br.

Exemplary compounds of the present invention include, but are not limited to:
5,5′,5″,5′″-(1,2,4,5-benzentetrayl)-tetrakis[4-pentyn-1-yl-(3-methylpyridinium)]tetrabromide;
5,5′,5″,5′″-(1,2,4,5-benzentetrayl)-tetrakis[4-pentyn-1-yl-(4-methylpyridinium)]tetrabromide;
5,5′,5″,5′″-(1,2,4,5-benzentetrayl)-tetrakis[4-pentyn-1-yl-(3,4-dimethylpyridinium)]tetrabromide;
5,5′,5″,5′″-(1,2,4,5-benzentetrayl)-tetrakis[4-pentyn-1-yl-(3,5-dimethylpyridinium)]tetrabromide;
5,5′,5″,5′″-(1,2,4,5-benzentetrayl)-tetrakis[4-pentyn-1-yl-nicotinium) tetrabromide;
5,5′,5″,5′″-(1,2,4,5-benzentetrayl]-tetrakis[4-pentyn-1-yl-(5,6,7,8-tetrahydroisoquinolinium)]tetrabromide;
5,5′,5″,5′″-(1,2,4,5-benzentetrayl)-tetrakis[4-pentyn-1-yl-(3-phenyl-pyridinium)]tetrabromide;
5,5′,5″,5′″-(1,2,4,5-benzentetrayl)-tetrakis(4-pentyn-1-yl-isoquinolinolinium) tetrabromide;
5,5′,5″,5′″-(1,2,4,5-benzentetrayl)-tetrakis[4-pentyn-1-yl-(3-benzyl-pyridinium)]tetrabromide;
5,5′,5″,5′″-(1,2,4,5-benzentetrayl)-tetrakis {4-pentyn-1-yl-[3-(3-hydroxypropyl)-pyridinium]}tetrabromide;
5,5′,5″,5′″-(1,2,4,5-benzentetrayl)-tetrakis[pentanyl-(3-methylpyridinium)]tetrabromide;
5,5′,5″,5′″-(2,4,5-benzentetrayl)-tetrakis[pentanyl-(4-methylpyridinium)]tetrabromide;
5,5′,5″,5′″-(1,2,4,5-benzentetrayl)-tetrakis[pentanyl-(4-dimethylpyridinium)]tetrabromide;
5,5′,5″,5′″-(1,2,4,5-benzentetrayl-tetrakis[pentanyl-(4-dimethylpyridinium)]tetrabromide;
5,5′,5″,5′″-(1,2,4,5-benzentetrayl)-tetrakis[pentanyl-4-nicotinium)]tetrabromide;
5,5′,5″,5′″-(1,2,4,5-benzentetrayl)-tetrakis[pentanyl-(3-(3-hydroxypropanyl)pyridinium)]tetrabromide;
5,5′,5″,5′″-(1,2,4,5-benzentetrayl)-tetrakis[pentanyl-4 isoquinolinium)]tetrabromide;
5,5′,″,5′″-(1,2,4,5-benzentetrayl)-tetrakis[pentanyl-(3-benzylpyridinium)]tetrabromide;
5,5′,5″,5′″-(1,2,4,5-benzentetrayl)-tetrakis[pentanyl-(3-phenylpyridinium)]tetrabromide; and
5,5′,5″,5′″-(1,2,4,5-benzentetrayl)-tetrakis[pentanyl-(5,6,7,8-tetrahydroisoquinolinium)]tetrabromide.

The compounds of the present invention may contain one or more stereocenters, and includes all possible diastereomers and all enantiomeric forms as well as racemic mixtures. The compounds may be separated into substantially optically pure compounds. The compounds of the present invention may be prepared by any applicable method known by those of skill in the art, including synthesis from corresponding free bases by reaction with an appropriate alkyl bromide. Tetrakis quaternary ammonium salts of the present invention and their mechanisms are also described in U.S. application Ser. No. 12/260,502, the contents of which are herein expressly incorporated herein by reference

The present invention provides a method for selectively modulating the function of a nicotinic acetylcholine receptor comprising administering a therapeutically effective amount of a compound as described above to a mammalian subject in need thereof. The present invention also provides a method of modulating, preventing, treating and/or reversing acute, chronic or cancer pain of central and/or peripheral origin that is referred to as nociceptive, neuropathic, visceral, inflammatory or somatic in nature comprising administering a therapeutically effective amount of a compound as described above to a mammalian subject in need thereof.

The present invention uses the compounds set forth herein in the treatment of any type of pain. Pain from nervous system disorders of central and/or peripheral origin, which may be treated according to the method of the present invention and includes any disorders involving pain including those types of pain referred to as nociceptive, neuropathic, inflammatory, somatic and visceral as well as acute, chronic, cancer-related, and surgical, as well as pain resulting from any and all injuries, diseases or toxin induced injuries of the central or peripheral nervous systems including pain accompanying stroke, multiple sclerosis, parkinson's disease and pain from peripheral neuropathy as a result of diabetes, HIV/AIDS, chemotherapeutic drugs, and/or alcohol. Pain from cancer, which may have its origin at any peripheral or central site, may be caused by tumor invasion of bone, tissue or nerve.

The present invention further provides a method for treating and/or preventing is inflammatory pain disorders comprising administering to a mammalian subject in need thereof a therapeutically effective amount of a compound of the present invention. Inflammatory pain disorders which may be treated according to the method of the present invention include ankylosing spondylitis, benign prostatic hyperplasia, cholecystitis, ulcerative colitis, Crohn's disease, diabetes mellitus, gastritis, glomerulonephritis, irritable bowel syndrome, multiple sclerosis, osteoarthritis, pancreatitis, polymyositis, psoriasis and rheumatoid arthritis.

In yet another embodiment, the present invention is directed to a method for preventing pain, comprising administering to a mammalian subject in need thereof a therapeutically effective amount of a compound of the present invention In such a method, the compound of the present invention may reduce a pain response.

The compounds of the present invention can be delivered directly or in pharmaceutical compositions along with suitable carriers or excipients, as is well known in the art. For example, a pharmaceutical composition of the invention may include a conventional additive, such as a stabilizer, buffer, salt, preservative, filler, flavor enancer and the like, as known to those skilled in the art. Exemplary buffers may include phosphates, carbonates, citrates and the like. Exemplary preservatives may include EDTA, EGTA, BHA, BHT and the like.

An effective amount of such agents can readily be determined by routine experimentation, as can the most effective and convenient route of administration and the most appropriate formulation. Various formulations and drug delivery systems are available in the art. See, e.g., Gennaro, A. R., ed. (1995) Remington's Pharmaceutical Sciences.

Suitable routes of administration may include oral, rectal, transmucosal, nasal, or intestinal administration and parenteral delivery, including intramuscular, subcutaneous, intramedullary injections, as well as intrathecal, direct intraventricular, intravenous, intraperitoneal, intranasal, or intraocular injections. In addition, the agent or composition thereof may be administered sublingually or via a spray. The agent or composition thereof may be administered in a local rather than a systemic manner. For example, a suitable agent can be delivered via injection or in a targeted drug delivery system, such as a depot or sustained release formulation.

The pharmaceutical compositions of the present invention may be manufactured by any of the methods well-known in the art, such as by conventional mixing, dissolving, granulating, dragee-making, levigating, emulsifying, encapsulating, entrapping, or lyophilizing processes. As noted above, the compositions of the present invention can include one or more physiologically acceptable carriers such as excipients and auxiliaries that facilitate processing of active molecules into preparations for pharmaceutical use.

Proper formulation is dependent upon the route of administration chosen. For injection, for example, the composition may be formulated in aqueous solutions, preferably in physiologically compatible buffers such as Hanks' solution, Ringer's solution, or physiological saline buffer. For transmucosal or nasal administration, penetrants appropriate to the barrier to be permeated are used in the formulation. Such penetrants are generally known in the art. In a preferred embodiment of the present invention, the present compounds are prepared in a formulation intended for oral administration. For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject. The compounds may also be formulated in rectal compositions such as suppositories or retention enemas, e.g., containing conventional suppository bases such as cocoa butter or other glycerides.

Pharmaceutical preparations for oral use can be obtained as solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Also, wetting agents such as sodium dodecyl sulfate may be included.

Dragee cores are provided with suitable coatings. For this purpose, concentrated sugar solutions may be used, which may optionally contain gum arable, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations for oral administration include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In one embodiment, the compounds of the present invention can be administered transdermally, such as through a skin patch, or micro-needle patch, or topically. In one aspect, the transdermal or topical formulations of the present invention can additionally comprise one or multiple penetration enhancers or other effectors, including agents that enhance migration of the delivered compound. Transdermal or topical administration could be preferred, for example, in situations in which location specific delivery is desired.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or any other suitable gas. In the case of a pressurized aerosol, the appropriate dosage unit may be determined by providing a valve to deliver a metered amount. Capsules and cartridges of, for example, gelatin, for use in an inhaler or insufflator may be formulated. These typically contain a powder mix of the compound and a suitable powder base such as lactose or starch.

Compositions formulated for parenteral administration by injection, e.g., by bolus injection or continuous infusion can be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing and/or dispersing agents. Formulations for parenteral administration include aqueous solutions or other compositions in water-soluble form.

Suspensions of the active compounds may also be prepared as appropriate oily injection suspensions. Suitable lipophilic solvents or vehicles include fatty oils such as sesame oil and synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Alternatively, the active ingredient may be in powder form for constitution with a suitable vehicle, e.g., sterile pyrogen-free water, before use.

As mentioned above, the compositions of the present invention may also be formulated as a depot preparation. Such long acting formulations may be administered by implantation (for example, subcutaneously or intramuscularly) or by intramuscular injection. Thus, for example, the present compounds may be formulated with suitable polymeric or hydrophobic materials (for example as an emulsion in an acceptable oil) or ion exchange resins, or as sparingly soluble derivatives, for example, as a sparingly soluble salt.

Suitable carriers for the hydrophobic molecules of the invention are well known in the art and include co-solvent systems comprising, for example, benzyl alcohol, a nonpolar surfactant, a water-miscible organic polymer, and an aqueous phase. The co-solvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system is effective in dissolving hydrophobic compounds and produces low toxicity upon systemic administration. Naturally, the proportions of a co-solvent system may be varied considerably without destroying its solubility and toxicity characteristics. Furthermore, the identity of the co-solvent components may be varied. For example, other low-toxicity nonpolar surfactants may be used instead of polysorbate 80, the fraction size of polyethylene glycol may be varied, other biocompatible polymers may replace polyethylene glycol, e.g., polyvinyl pyrrolidone, and other sugars or polysaccharides may substitute for dextrose.

Alternatively, other delivery systems for hydrophobic molecules may be employed. Liposomes and emulsions are well known examples of delivery vehicles or carriers for hydrophobic drugs. Liposomal delivery systems are discussed above in the context of gene-delivery systems. Certain organic solvents such as dimethylsulfoxide also may be employed, although usually at the cost of greater toxicity. Additionally, the compounds may be delivered using sustained-release systems, such as semi-permeable matrices of solid hydrophobic polymers containing the effective amount of the composition to be administered. Various sustained-release materials are established and available to those of skill in the art. Sustained-release capsules may, depending on their chemical nature, release the compounds for a few weeks up to over 100 days. Depending on the chemical nature and the biological stability of the therapeutic reagent, additional strategies for stabilization may be employed.

For any composition used in the present methods of treatment, a therapeutically effective dose can be estimated initially using a variety of techniques well known in the art. Dosage ranges appropriate for human subjects can be determined, for example, using data obtained from animal studies.

A therapeutically effective dose of an agent refers to that amount of the agent that results in amelioration of symptoms. Toxicity and therapeutic efficacy of such molecules can be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio of toxic to therapeutic effects is the therapeutic index, which can be expressed as the ratio LD₅₀/ED₅₀. Agents that exhibit high therapeutic indices are preferred.

Dosages preferably fall within a range of circulating concentrations that includes the ED₅₀ with little or no toxicity. Dosages may vary within this range depending upon the dosage form employed and the route of administration utilized. The exact formulation, route of administration, and dosage should be chosen, according to methods known in the art, in view of the specifics of a subject's condition.

The amount of agent or composition administered will, of course, be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

These and other embodiments of the present invention will readily occur to those of ordinary skill in the art in view of the disclosure herein, and are specifically contemplated.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications fall within the scope of the appended claims.

### Example 1

### Preparation of 1,2,4,5-tetraiodobenzene

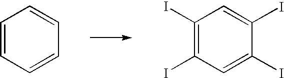

Periodic acid (2.56 g, 11.2 mmol) was dissolved with stirring in concentrated H₂SO₄ (60 mL) Potassium iodide (5.58 g, 33.6 mmol) was crushed and added to the clear solution. After about 30 min of stirring, the dark mixture was placed in an ice bath. The aromatic substrate (C₆H₅, 1 mL, 11.2 mmol) was then added slowly. The reaction was allowed to stir to room temperature for 1 day and poured onto crushed ice. The resulting solid was collected by suction filtration and washed well with methanol to remove iodine. The crude lavender powder (5.4 g 82% yield) was crystallized from 2-methoxyethanol, giving 1,2,4,5-tetraiodobenzene (71% yield) as white needles, mp 252-255° C. ¹H NMR (Me₂SO-d6) δ 8.32 (s); ¹³C NMR (Me₂SO-d6) 147.1, 108.5 ppm.

### Example 2

### Preparation of 5,5′,5″,5′″-(1,2,4,5-benzentetrayl)-tetrakis(4-pentyn-ol)

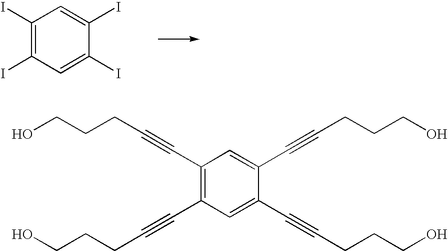

To a degassed solution of 1,2,4,5-tetraiodobenzene (5.81 g, 0.01 mol) in DMF-Et₃N (100 mL, 1:1) were added Pd(PPh₃)₂Cl₂ (350 mg. 0.5 mmol), CuI (200 mg, 1.2 mmol), and 4-pentyn-1-ol (4.2 g, 0.05 mol) was added drop-wise. The mixture was stirred under N₂ at room temperature for 24 h. The solution was poured into water (400 mL). The mixture was extracted with CH₂Cl₂ (3×200 mL). The combined organic phases were washed with 5% HCl and brine, dried over Na₂SO₄, and concentrated under vacuum. The residue was purified by silica gel column chromatography using CH₂Cl₂-MeOH (10:1, v/v) as eluent to afford tetramer (3.34 g, 82%): ¹H NMR (300 MHz, CD₃Cl+CO₃OD δ ppm), 7.30 (s, 2H), 3.72 (t, J=6.3 Hz, 8H), 2.52 (t, J=7.2 Hz, 8H), 1.80 (p, J=6.6 Hz, 8H) ppm.

### Example 3

### Preparation of 5,5′,5″,5′″-(1,2,4,5-benzentetrayl)-tetrakis(1-bromo-4-pentyne)

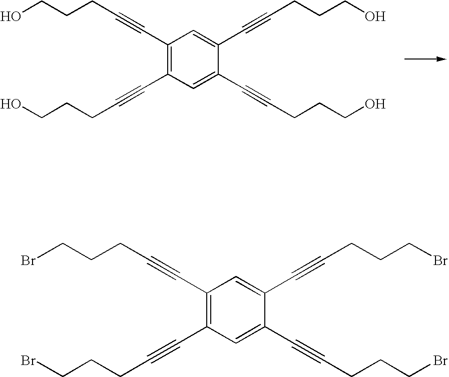

5,5′,5″,5′″-(1,2,4,5-benzentetrayl)tetrakis-4-pentyn-ol (2.05 g, 5.03 mmol) and carbon tetrabromide (7.41 g, 22.35 mmol) were dissolved in dry methylene chloride (100 mL) and cooled to 0° C. Triphenyl phosphine (6.16 g, 23.47 mmol) was added portion-wise and the mixture was stirred at RT. After the starting alcohol was consumed methanol was added and the mixture was stirred for an additional 5 minutes. The mixture concentrated and was treated with hexanes (500 mL) and then filtered through a short silica gel column, washed with ethylacetate/hexanes (1/4). The combined organic solvents were evaporated to dryness under reduced pressure. The resulting residue was purified by column chromatography (hexanes) to afford 2.84 g of the title compound. Yield: 89%. ¹H NMR (300 MHz, CDCl₃) δ 7.39 (s, 2H), 3.62 (t, J=6.3 Hz, 8H), 2.67 (t, J=6.6 Hz, 8H), 1.14 (p, J=6.6 Hz, 8H) ppm.

### Example 4

### Preparation of 5,5′,5″,5′″-(1,2,4,5-benzentetrayl)-tetrakis-(pentan-1-ol)

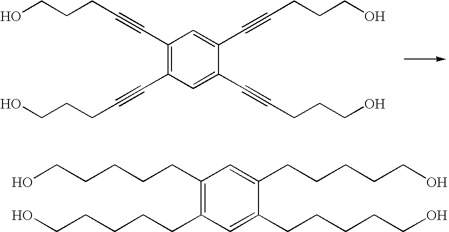

5,5′,5″,5′″-(1,2,4,5-benzentetrayl)tetrakis-4-pentyn-1-ol (2.01 g, 4.95 mmol) was dissolved in methanol (30 mL) and 10% Pd/C (5% w/w) was added. The resulting mixture was hydrogenated on a Parr hydrogenation apparatus (45 psig) for 4 hrs. The catalyst was removed by filtration through a Celite pad. The filter cake was rinsed with methanol, and the combined organic liquors were concentrated under reduced pressure. The crude product was purified by column chromatography (CHCl₃:MeOH, 10:1) to afford 1.97 g of the title compound. Yield: 95%. ¹H NMR (300 MHz, CDCl₃) δ 6.81 (s, 3H), 3.62 (t, J=6.3 Hz, 6H), 2.57 (t, J=7.5 Hz, 6H), 1.53-1.70 (m, 12H), 1.38 (m, 6H) ppm; ¹³C NMR (75 MHz, CDCl₃) δ 142.5, 126.1, 63.1, 36.1, 32.9, 31.5, 25.7 ppm.

### Example 5

### Preparation of 5′,5″,5′″-(1,2,4,5-benzentetrayl)-tetrakis(1-bromopentane)

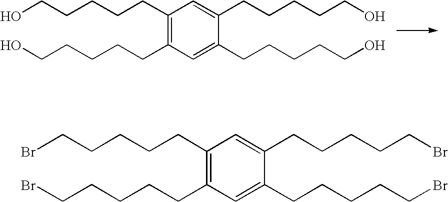

5,5′,5″,5′″-(1,2,4,5-benzentetrayl)-tetrakis-pentan-1-ol (1.97 g, 4.67 mmol) and carbon tetrabromide (7.22 g, 21.74.80 mmol) were dissolved in dry methylene chloride (50 mL) and cooled to 0° C. Triphenyl phosphine (5.70 g, 22.02 mmol) was added portion-wise and the mixture was stirred at RT. After the starting alcohol was consumed methanol was added and the mixture was stirred for an additional 5 minutes. The mixture concentrated and was treated with hexanes (500 mL) and then filtered through a short silica gel column, and washed with ethylacetate/hexanes (1/4). The combined organic solvents were evaporated to dryness under reduced pressure. The resulting residue was purified by column chromatography (hexanes) to afford 2.83 g of the title compound. Yield: 90%. ¹H NMR (300 MHz, CDCl₃) δ 6.89 (s, 2H), 3.42 (t, J=7.2 Hz, 8H), 2.55 (m, 8H), 1.90 (m, 8H), 1.45-1.62 (m, 16H) ppm.

### Example 6

### Preparation of 5,5′,5″,5′″-(1,2,4,5-benzentetrayl)-tetrakis[4-pentyn-1-yl-(3-methylpyridinium)]tetrabromide

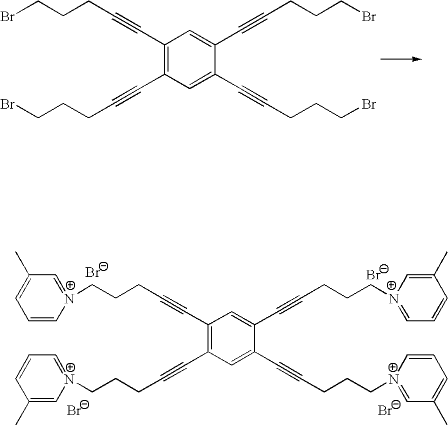

A mixture of 5,5′,5″,5′″-(1,2,4,5-benzentetrayl)tetrakis-1-bromo-4-pentyne (300 mg, 0.46 mmol) and 3-picoline (607 mg, 6.52 mmol) was heated at 60-70° C. for 12 hrs. The resulted mixture was treated with diethyl ether and then dissolved in water (15 mL), the aqueous solution was extracted extensively with chloroform (30 mL×5). Water was removed by lyophilization to afford 385 mg of the title compound. Yield: 82%. ¹H NMR (300 MHz, CD₃OD) δ 9.01 9.11 (s, 4H), 9.01 (d, J=6 Hz, 4H), 8.38 (d, J=8.1 Hz, 4H), 8.01 (t, J1=6 Hz, J2=8.1 Hz, 4H), 7.41 (s, 2H), 4.88 (t, J=8.4 Hz, 8H), 2.74 (t, J=6.6 Hz, 8H), 2.57 (s, 12H), 2.40 (p, J=6.6 Hz, 8H) ppm.

### Example 7

### Preparation of 5,5′,5″,5′″-(1,2,4,5-benzentetrayl)-tetrakis[4-pentyn-1-yl-(4-methylpyridinium)]tetrabromide

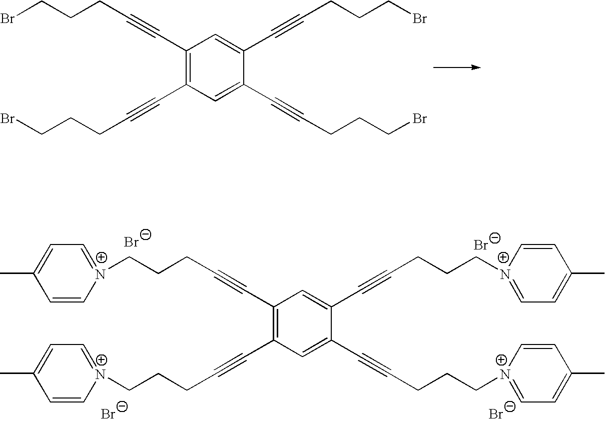

A mixture of 5,5′,5″,5′″-(1,2,4,5-benzentetrayl)tetrakis-1-bromo-4-penyne (300 mg, 0.46 mmol) and 4-picoline (600 mg, 6.50 mmol) was heated at 60-70° C. for 12 hrs. The resulted mixture was treated with diethyl ether and then dissolved in water (15 mL), the aqueous solution was extracted extensively with chloroform (30 mL×5). Water was removed by lyophilization to afford 408 mg of the title compound. Yield: 87%. ¹H NMR (300 MHz, CD₃OD) δ ppm 8.95 (d, J=6.0 Hz, 8H), 7.92 (d, J=6.0 Hz, 8H), 7.37 (s, 2H), 4.82 (t, J=6.3 Hz, 8H), 2.71 (t, J=6.6 Hz, 8H), 2.58 (s, 12H), 2.35 (p, J=6.6 Hz, 8H) ppm.

### Example 8

### Preparation of 5,5′,5″,5′″-(1,2,4,5-benzentetrayl)-tetrakis[4-pentyn-1-yl-(3,4-dimethylpyridinium)]tetrabromide

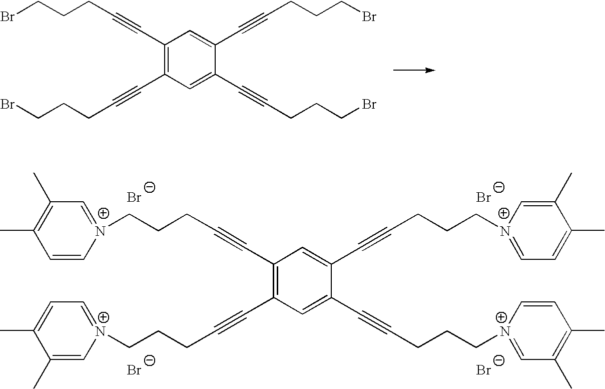

A mixture of 5,5′,5″,5′″-(1,2,4,5-benzentetrayl)tetrakis-1-bromo-4-pentyne (300 mg, 0.46 mmol) and 3,4-lutidine (650 mg, 5.0 mmol) was heated at 60-70° C. for 12 hrs. The resulted mixture was treated with diethyl ether and then dissolved in water (15 mL), the aqueous solution was extracted extensively with chloroform (30 mL×5). Water was removed by lyophilization to afford 374 mg of the title compound. Yield: 75%. ¹H NMR (300 MHz, CD₃OD) δ ppm 8.91 (s, 4H), 8.80 (d, J=6.0 Hz, 4H), 7.85 (d, J=6.6 Hz, 4H), 7.32 (s, 2H), 4.78 (t, J=6.6 Hz, 8H), 2.71 (t, J=6.3 Hz, 8H), 2.46 (s, 12H), 2.43 (s, 12H), 2.35 (t, J=6.6 Hz, 8H) ppm.

### Example 9

### Preparation of 5,5′,5″,5′″-(1,2,4,5-benzentetrayl)-tetrakis[4-pentyn-1-yl-(3,5-dimethylpyridinium)]tetrabromide

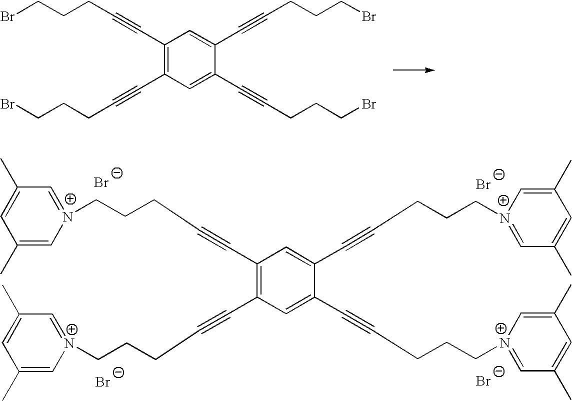

A mixture of 5,5′,5″,5′″-(1,2,4,5-benzentetrayl)tetrakis-1-bromo-4-pentyne (300 mg, 0.46 mmol) and 3,5-lutidine (650 mg, 5.0 mmol) was heated at 60-70° C. for 12 hrs. The resulted mixture was treated with diethyl ether and then dissolved in water (15 mL), the aqueous solution was extracted extensively with chloroform (30 mL×5). Water was removed by lyophilization to afford 425 mg of the title compound. Yield: 85%. ¹H NMR (300 MHz, CD₃OD) δ ppm 8.86 (s, 8H), 8.17 (s, 4H), 7.39 (s, 4H), 4.79 (t, J=7.1 Hz, 8H), 2.72 (t, J=6.6 Hz, 8H), 2.51 (s, 24H), 2.37 (t, J=6.6 Hz, 8H) ppm.

### Example 10

### Preparation of 5,5′,5″,5′″-(1,2,4,5-benzentetrayl)-tetrakis(4-pentyn-1-yl-nicotinium) tetrabromide

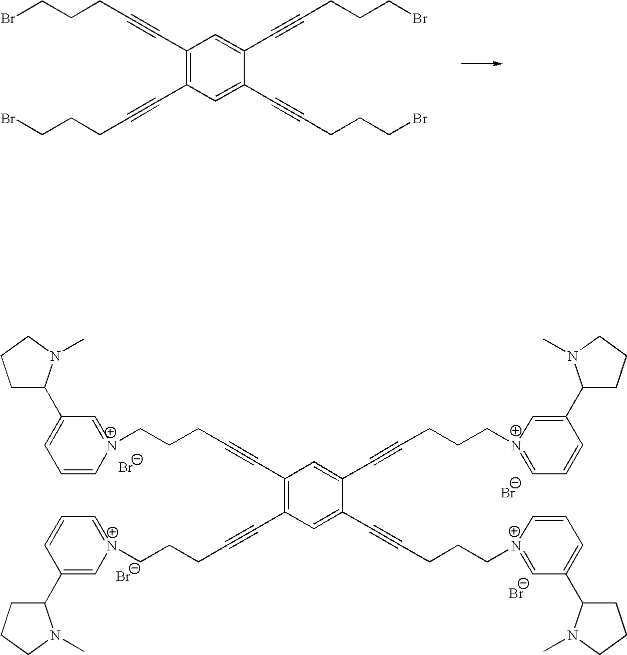

A mixture of 5,5′,5″,5′″-(1,2,4,5-benzentetrayl)tetrakis-1-bromo-4-pentyne (300 mg, 0.46 mmol) and S(−)-nicotine (320 mg, 2 mmol) in acetonitrile was heated at 60-70° C. for 24 hrs. The resulted mixture was treated with diethyl ether and then dissolved in water (15 mL), the aqueous solution was extracted extensively with chloroform (30 mL×5). Water was removed by lyophilization to afford 390 mg of the title compound. Yield: 60%. ¹H NMR (300 MHz, CD₃OD) δ 99.15 (s, 4H), 9.05 (d, J=6 Hz, 4H), 8.54 (d, J=7.8 Hz, 4H), 8.10 (t, J1=6 Hz, J2=7.8 Hz, 4H), 7.48 (s, 2H), 4.91 (t, J=7.5 Hz, 8H), 3.55 (t, J=8.1 Hz, 4H), 3.24 (m, 4H), 2.71 (t, J=8.1 Hz, 8H), 2.36-2.47 (m, 16H), 2.25 (s, 12H), 1.89-1.95 (m, 8H), 1.72-1.76 (m, 4H) ppm.

### Example 11

### Preparation of 5,5′,5″,5′″-(1,2,4,5-benzentetrayl)-tetrakis[4-pentyn-1-yl-(5,6,7,8-tetrahydroisoquinolinium)]tetrabromide

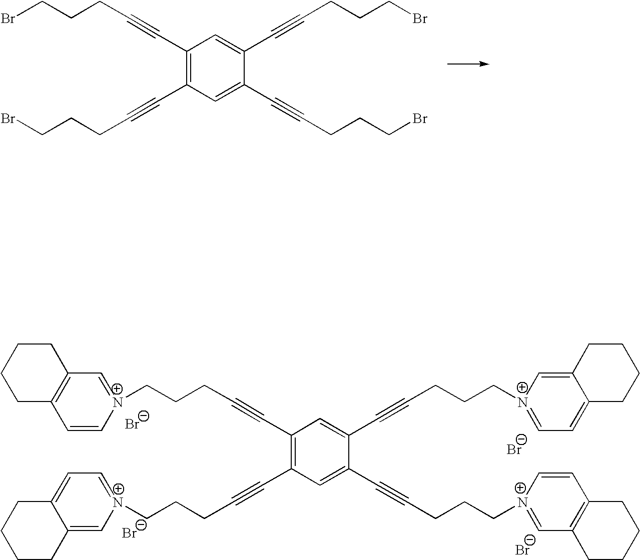

A mixture of 5,5′,5″,5′″-(1,2,4,5-benzentetrayl)tetrakis-1-bromo-4-pentyne (300 mg, 0.46 mmol) and 5,6,7,8-tetrahydroisoquinoline (260 mg, 2.0 mmol) was heated at 60-70° C. for 18 hrs. The resulted mixture was treated with diethyl ether and then dissolved in water (15 mL), the aqueous solution was extracted extensively with chloroform (30 mL×5). Water was removed by lyophilization to afford 320 mg of the title compound. Yield: 80%. ¹H NMR (300 MHz) δ (CD₃Cl), 8.86 (s, 4H), 8.72 (d, 4H), 7.75 (s, 4H), 7.30 (s, 2H), 4.74 (t, J=8.1 Hz, 8H), 2.91 (br, 16H), 2.72 (t, J=6.6 Hz, 8H), 2.35 (m, 8H), 1.80 (br, 16H). ¹³C NMR, 159.92, 145.40, 141.74, 140.03, 136.20, 129.26, 126.28, 95.18, 80.49, 61.50, 30.94, 30.56, 27.51, 22.30, 22.19, 17.73 ppm.

### Example 12

### Preparation of 5,5′,5″,5′″-(1,2,4,5-benzentetrayl)-tetrakis[4-pentyn-1-yl-(3-phenyl-pyridinium)]tetrabromide

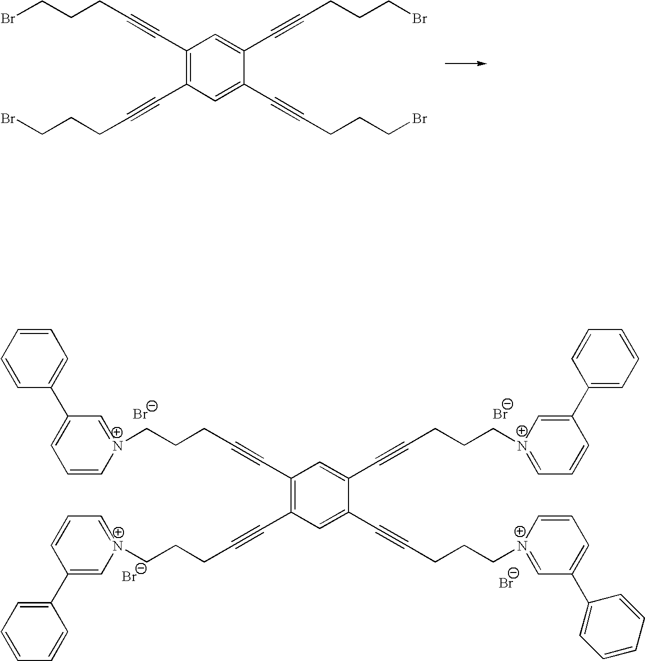

A mixture of 5,5′,5″,5′″-(1,2,4,5-benzentetrayl)tetrakis-1-bromo-4-pentyne (300 mg, 0.46 mmol) and 3-phenylpyridine (310 mg, 2.0 mmol) was heated at 60-70° C. for 18 hrs. The resulting mixture was treated with diethyl ether and then dissolved in water (15 mL), the aqueous solution was extracted extensively with chloroform (30 mL×5). Water was removed by lyophilization to afford 440 mg of the title compound. Yield: 75%. ¹H NMR (300 MHz, CD₃OD) δppm 9.50 (s, 4H), 9.09 (d, J=6.0 Hz, 4H), 8.73-8.76 (m, 4H), 7.12-7.16 (m, 4H), 7.76-7.81 (m, 8H), 7.52-7.57 (m, 12H), 7.23 (s, 2H), 4.95 (t, J=8.4 Hz, 8H), 2.71 (t, J=6.6 Hz, 8H), 2.40 (m, 8H) ppm. ¹³C NMR, 144.23, 142.61, 136.23, 134.47, 131.51, 130.78, 139.56, 128.70, 126.13, 94.82, 80.97, 62.58, 31.08, 17.68 ppm.

### Example 13

### Preparation of 5,5′,5″,5′″-(1,2,4,5-benzentetrayl)-tetrakis[4-pentyne-1-yl (isoquinolinolinium)]tetrabromide

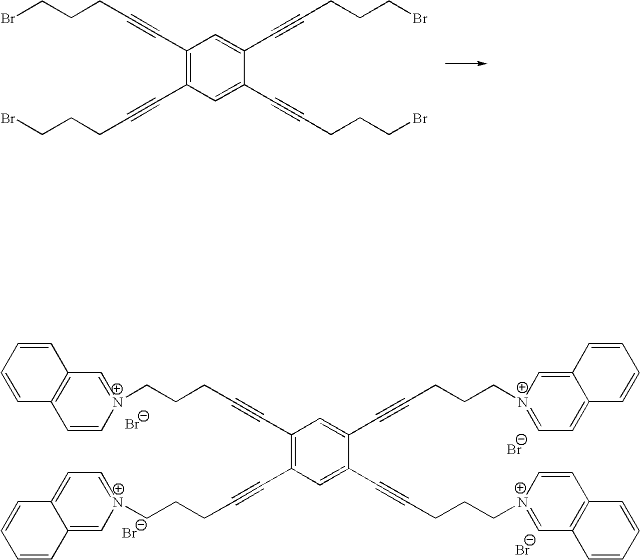

A mixture of 5,5′,5″,5′″-(1,2,4,5-benzentetrayl)tetrakis-1-bromo-4-pentyne (300 mg, 0.46 mmol) and isoquinoline (260 mg, 2.0 mmol) was heated at 60-70° C. for 18 hrs. The resulting mixture was treated with diethyl ether and then dissolved in water (15 mL), the aqueous solution was extracted extensively with chloroform (30 mL×5). Water was removed by lyophilization to afford 442 mg of the title compound. Yield: 82%. ¹H NMR (300 MHz, CD₃OD) δ 10.14 (s, 4H), 8.80 (d, J=6.6 Hz, 4H), 8.47 (d, J=7.5 Hz, *H), 8.147-8.17 (m, 8H), 8.00 (m, 4H), 6.59 (s, 2H), 5.02 (t, J=6.6 Hz, 8H), 2.79 (t, J=6.3 Hz, 8H), 2.44-2.50 (m, 8H) ppm. ¹³C NMR, 144.23, 142.61, 136.23, 134.48, 131.51, 130.78, 129.56, 128.70, 126.13, 94.82, 80.97, 62.57, 31.08, 17.68 ppm.

### Example 14

### Preparation of 5,5′,5″,5′″-(1,2,4,5-benzentetrayl)-tetrakis[4-pentyn-1-yl-(3-benzyl-pyridinium)]tetrabromide

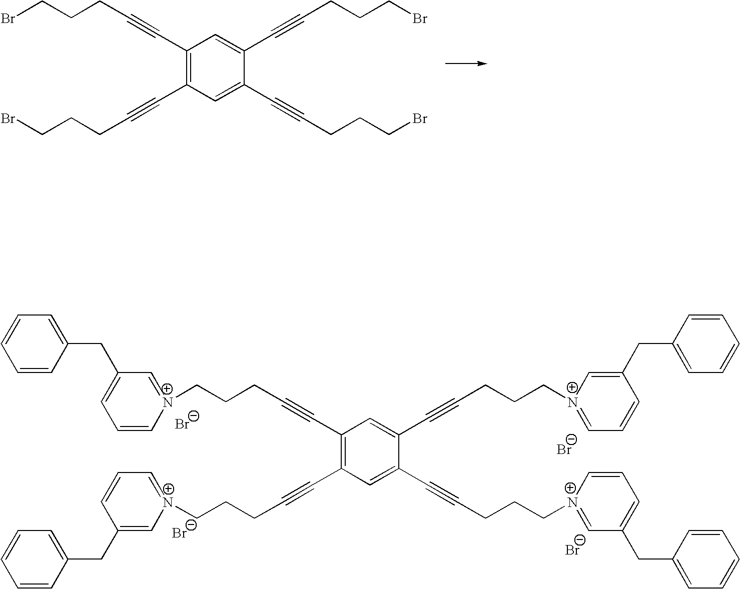

A mixture of 5,5′,5″,5′″-(1,2,4,5-benzentetrayl)tetrakis-1-bromo-4-pentyne (300 mg, 0.46 mmol) and 3-benzyl pyridine (340 mg, 2.0 mmol) was heated at 60-70° C. for 18 hrs. The resulting mixture was treated with diethyl ether and then dissolved in water (15 mL), the aqueous solution was extracted extensively with chloroform (30 mL×5). Water was removed by lyophilization to afford 521 mg of the title compound. Yield: 85%. ¹H NMR (300 MHz, CD₃OD) δ 9.21 (s, 4H), 9.02 (d, 4H), 8.26 (d, 4H), 8.00 (dd, 4H), 7.42 (s, 2H), 7.18-7.28 (m, 20H), 4.86 (t, 8H), 4.22 (s, 8H), 2.67 (t, 8H), 2.34-2.44 (m, 8H) ppm.

### Example 15

### Preparation of 5,5′,5″,5′″-(1,2,4,5-benzentetrayl)-tetrakis {4-pentyn-1-yl-[3-(3-hydroxypropyl)-pyridinium]) tetrabromide

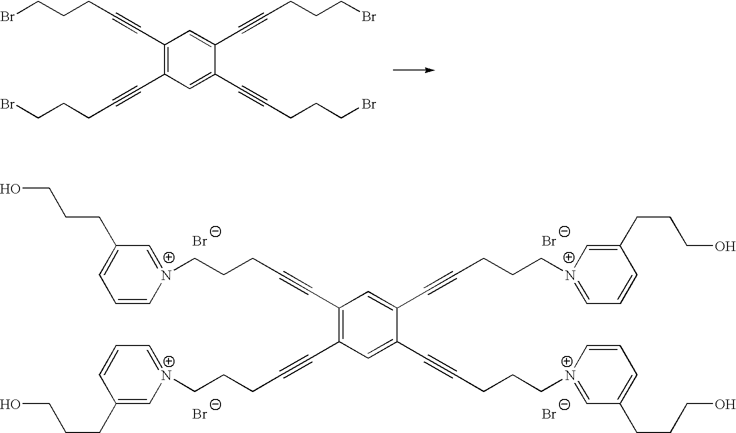

A mixture of 5,5′,5″,5′″-(1,2,4,5-benzentetrayl)tetrakis-[1-bromo-4-pentyne] (300 mg, 0.46 mmol) and 3-hydroxypropylpyridine (340 mg, 2.0 mmol) was heated at 60-70° C. for 18 hrs. The resulting mixture was treated with diethyl ether and then dissolved in water (15 mL), the aqueous solution was extracted extensively with chloroform (30 mL×5). Water was removed by lyophilization to afford 350 mg of the title compound. Yield: 63%. ¹H NMR (300 MHz, CD₃OD) δ 9.14 (s, 4H), 9.04 (d, J=6.0 Hz, 4H), 8.45 (d, J=8.1 Hz, 4H), 8.05 (dd, J1-6.0 Hz, J2-8.1 Hz, 4H), 7.39 (s, 2H), 4.90 (t, J=6.9 Hz, 8H), 3.61 (t, J=6.0 Hz, 8H), 2.96 (t, J=7.8 Hz, 8H), 2.75 (t, J=6.6 Hz, 8H), 2.36-2.45 (m, 8H), 1.88-1.97 (m, 8H) ppm.

### Example 16

### Preparation of 5,5′,5″,5′″-(1,2,4,5-benzentetrayl)-tetrakis[pentanyl-(3-methyl-pyridinkim)]tetrabromide

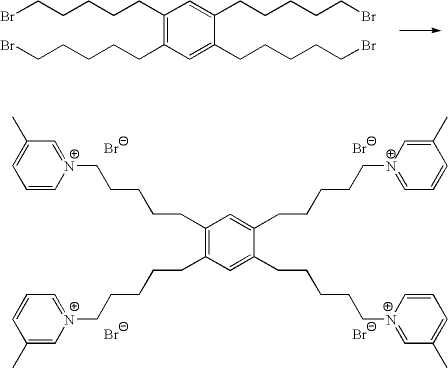

A mixture of 5,5′,5″,5′″-(1,2,4,5-benzentetrayl)tetrakis[1-bromopentane] (330 mg, 0.49 mmol) and 3-picoline (220 mg, 2.3 mmol) was heated at 60-70° C. for 18 hrs. The resulting mixture was treated with diethyl ether and then dissolved in water (15 mL), the aqueous solution was extracted extensively with chloroform (30 mL×5). Water was removed through lyophilization to afford 400 mg of the title compound. Yield: 78%. ¹H NMR (300 MHz, CD₃OD) δ ppm 9.08 (s, 4H), 8.96 (d, J=6.0 Hz, 4H), 8.47 (d, J=8.1 Hz, 4H), 8.02 (dd, J1=8.1 Hz, J2=6.0 Hz, 4H), 6.92 (s, 2H), 4.70 (t, J=7.8 Hz, 8H), 2.61 (s, 12H), 2.58 (t, J=7.8 Hz, 8H), 2.08-2.14 (m, 8H), 1.50-1.65 (m, 8H), 1.45-1.52 (m, 8H) ppm. ¹³C NMR, 146.10, 144.43, 141.97, 139.95, 137.35, 130.17, 127.60, 61.71, 32.07, 31.54, 31.09, 26.24, 17.79 ppm.

### Example 17

### Preparation of 5,5′,5″,5′″-(1,2,4,5-benzentetrayl)-tetrakis[pentanyl-(4-methyl-pyridinium)]tetrabromide

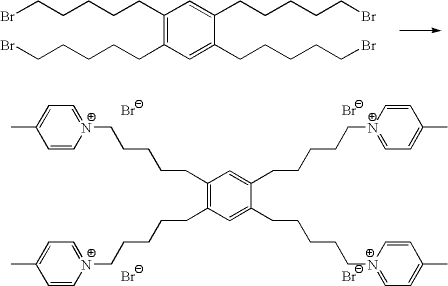

A mixture of 5,5′,5″,5′″-(1,2,4,5-benzentetrayl)tetrakis-[1-bromopentane] (330 mg, 0.49 mmol) and 4-picoline (220 mg, 2.3 mmol) was heated at 60-70° C. for 18 hrs. The resulting mixture was treated with diethyl ether and then dissolved in water (15 mL), the aqueous solution was extracted extensively with chloroform (30 mL×5). Water was removed through lyophilization to afford 425 mg of the title compound. Yield: 83%. ¹H NMR (300 MHz, CD₃OD) δ 8.97 (d, J=6.6 Hz, 8H), 7.97 (d, J=6.6 Hz, 8H), 6.91 (s, 2H), 4.67 (t, J=7.5 Hz, 8H), 2.67 (s, 12 Hz), 2.57 (t, J=7.5 Hz, 8H), 2.04-2.11 (m, 8H), 1.57-1.65 (m, 8H), 1.46-1.50 (m, 8H) ppm. ¹³C NMR, 159.69, 143.48, 137.35, 130.17, 128.79, 61.01, 32.05, 31.42, 31.09, 26.17 ppm.

### Example 18

### Preparation of 5,5′,5″,5′″-(1,2,4,5-benzentetrayl)-tetrakis[pentanyl-(3,4-dimethylpyridinium)]tetrabromide

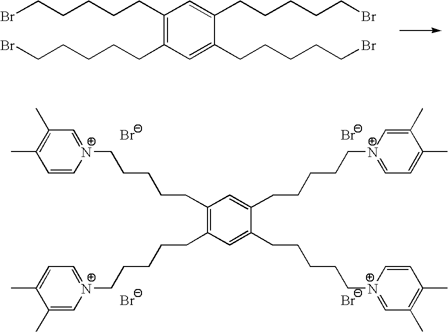

A mixture of 5,5′,5″,5′″-(1,2,4,5-benzentetrayl)tetrakis-[1-bromopentane] (330 mg, 0.49 mmol) and 3,4-lutidine (220 mg, 2.3 mmol) was heated at 60-70° C. for 18 hrs. The resulting mixture was treated with diethyl ether and then dissolved in water (15 mL), the aqueous solution was extracted extensively with chloroform (30 mL×5). Water was removed through lyophilization to afford 421 mg of the title compound. Yield: 78%. ¹H NMR (300 MHz, CD₃OD) δ 8.84 (s, 4H), 8.73 (d, J=6.3 Hz, 4H), 7.86 (d, J=6.3 Hz, 4H0, 6.89 (s, 2H), 4.57 (t, J=7.5 Hz, 8H), 2.58 s, 12H), 2.56 (t, J=7.5 Hz, 8H), 2.48 (s, 12H), 2.01-2.08 (m, 8H), 1.56-1.64 (m, 8H), 1.42-1.49 (m, 8H) ppm. ¹³C NMR, 158.40, 142.97, 141.35, 138.69, 137.28, 130.11, 128.27, 60.85, 32.04, 31.41, 31.08, 26.22, 15.94 ppm.

### Example 19

### Preparation of 5,5′,5″,5′″-(1,2,4,5-benzentetrayl)-tetrakis[pentanyl-(3,5-dimethylpyridinium)]tetrabromide

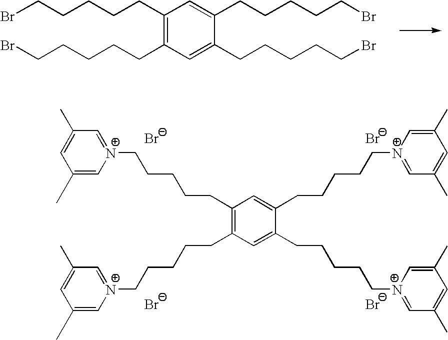

A mixture of 5,5′,5″,5′″-(1,2,4,5-benzentetrayl)tetrakis-[1-bromopentane] (330 mg, 0.49 mmol) and 3,5-lutidine (220 mg, 2.3 mmol) was heated at 60-70° C. for 18 hrs. The resulting mixture was treated with diethyl ether and then dissolved in water (15 mL), the aqueous solution was extracted extensively with chloroform (30 mL×5). Water was removed through lyophilization to afford 475 mg of the title compound. Yield: 88%. ¹H NMR (300 MHz, CD₃OD) δ 8.76 (s, 8H), 8.25 (s, 6.90 (s, 2H), 4.57 (t, J=7.8 Hz, 8H), 2.57 (t, J=7.5 Hz, 2.53 (s, 12H), 2.03-2.08 (m, 8H), 2.58-1.64 (m, 8H), 1.45-1.50 (m, 8H) ppm. ¹³C NMR, 147.72, 142.75, 140.37, 138.52, 131.34, 62.77, 33.27, 32.73, 32.34, 27.53, 18.55 ppm.

### Example 20

### Preparation of 5,5′,5″,5′″-(1,2,4,5-benzentetrayl)-tetrakis[pentanyl-(nicotinium)]tetrabromide

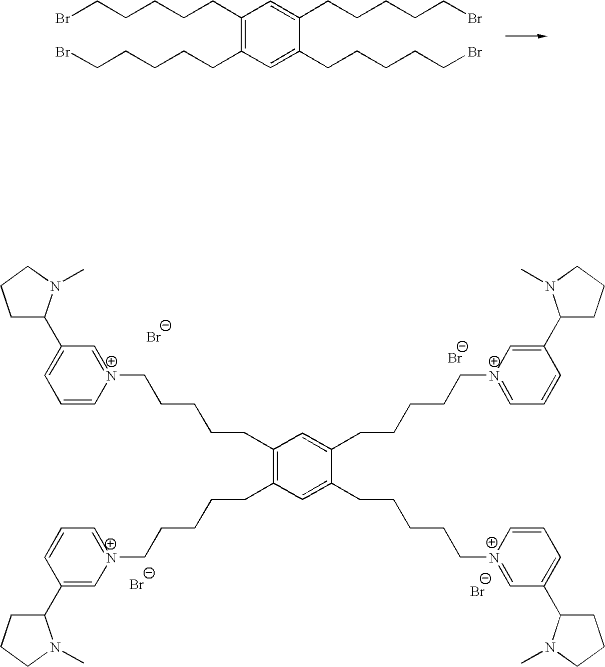

A mixture of 5,5′,5″,5′″-(1,2,4,5-benzentetrayl)tetrakis[1-bromopentane] (330 mg, 0.49 mmol) and S-(−)-nicotine (355 mg, 2.3 mmol) was heated at 60-70° C. for 18 hrs. The resulted mixture was treated with diethyl ether and then dissolved in water (15 mL), the aqueous solution was extracted extensively with chloroform (30 mL×5). Water was removed through lyophilization to afford 362 mg of the title compound. Yield: 56%. ¹H NMR (300 MHz, CD₃OD) δ 9.06 (s, 4H), 8.96 (d, J=6.6 Hz, 4H), 8.57 (d, J=8.1 Hz, 4H), 8.07 (dd, 4H), 6.89 (s, 2H), 4.69 (t, J=7.5 Hz, 8H), 3.54 (m, 4H), 3.24-3.34 (m, 4H), 2.38-2.59 (8H), 2.24 (s, 12H), 1.90-2.11 (m, 16 Hz), 1.74-1.78 (m, 4H), 1.61 (br, 8H), 1.48 (br, 8H) ppm. ¹³C NMR, 145.80, 144.43, 143.72, 143.44, 137.28, 128.12, 67.45, 61.89, 56.79, 39.69, 35.14, 32.05, 31.55, 31.06, 26.25, 22.93 ppm.

### Example 21

### Preparation of 5,5′,5″,5′″-(1,2,4,5-benzentetrayl)-tetrakis[pentanyl-(3-(3-hydroxypropanyl)pyridinium)]tetrabromide

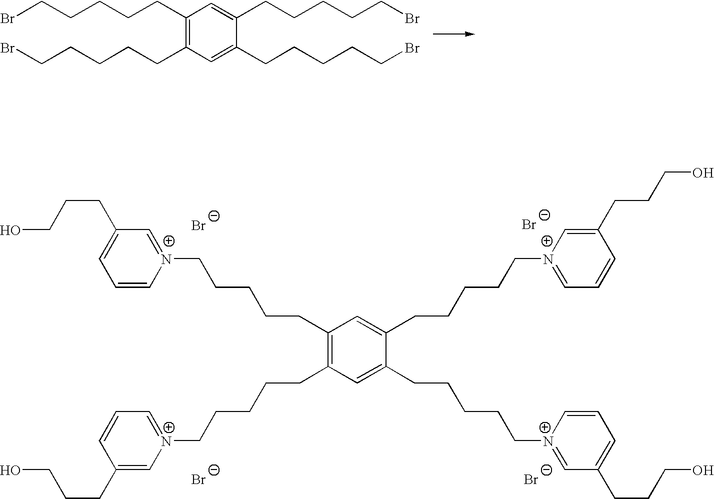

A mixture of 5,5′,5″,5′″-(1,2,4,5-benzentetrayl)tetrakis-[1-bromopentane] (330 mg, 0.49 mmol) and 3-(3-hydroxypropanyl)-pyridine (325 mg, 2.3 mmol) was heated at 60-70° C. for 18 hrs. The resulting mixture was treated with diethyl ether and then dissolved in water (15 mL), the aqueous solution was extracted extensively with chloroform (30 mL×5). Water was removed through lyophilization to afford 371 mg of the title compound. Yield: 62%. ¹H NMR (300 MHz, CD₃OD) δ 9.07 (s, 4H), 8.94 (d, J=6.0 Hz, 4H), 8.50 (d, J=8.4 Hz, 4H), 8.05 (dd, 4H), 6.90 (s, 2H), 4.69 (t, J=7.5 Hz, 8H), 3.62 (t, J=6.0 Hz, 8H), 2.99 (t, 7.8 Hz, 8H), 2.57 (t, J=7.5 Hz, 8H), 2.05-2.12 (m, 8H), 1.93-1.98 (m, 8H), 1.57-1.62 (m, 8H), 1.46-1.50 (m, 8H) ppm. ¹³C NMR, 145.53, 144.24, 143.98, 142.24, 137.30, 130.15, 127.80, 61.77, 60.48, 33.08, 32.05, 31.55, 31.06, 29.12, 26.20 ppm.

### Example 22

### Preparation of 5,5′,5″,5′″-(1,2,4,5-benzentetrayl)-tetrakis[pentanyl-(isoquinolinium)]tetrabromide

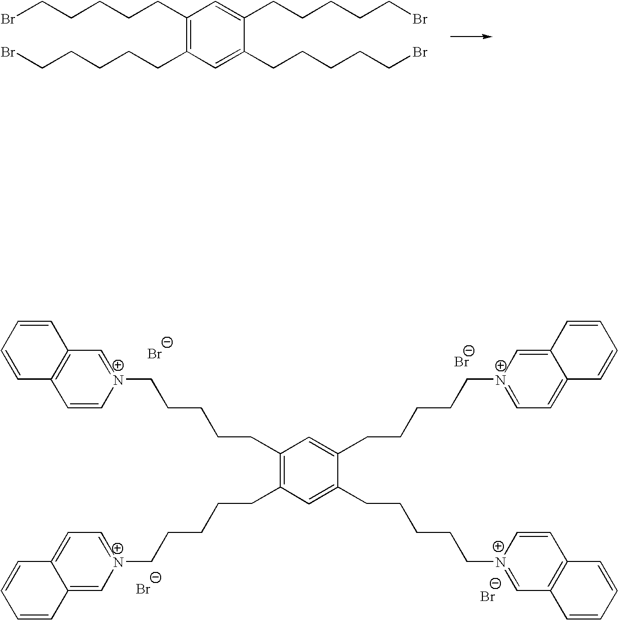

A mixture of 5,5′,5″,5′″-(1,2,4,5-benzentetrayl)-tetrakis-[1-bromopentane] (330 mg, 0.49 mmol) and 3-(3-hydroxypropanyl)-pyridine (300 mg, 2.3 mmol) was heated at 60-70° C. for 18 hrs. The resulted mixture was treated with diethyl ether and then dissolved in water (15 mL), the aqueous solution was extracted extensively with chloroform (30 mL×5). Water was removed through lyophilization to afford 460 mg of the title compound. Yield: 79%. ¹H NMR (300 MHz, CD₃OD) δ 10.15 (s, 4H), 8.78-8.81 (m, 4H), 8.49-8.54 (m, 8H), 8.27-8.32 (m, 4H), 8.17-8.28 (m, 4H), 8.00-8.05 (m, 4H), 6.80 (s, 2H), 4.87 (t, J=7.5 Hz, 8H), 2.44-2.51 (m, 8H), 2.15-2.22 (m, 8H), 1.46-1.58 (m, 16H) ppm. ¹³C NMR 149.62, 137.62, 137.18, 137.12, 134.77, 131.40, 130.42, 130.07, 127.84, 127.39, 126.38, 61.72, 31.97, 31.38, 30.94, 26.23 ppm.

### Example 23

### Preparation of 5,5′,5″,5′″-(1,2,4,5-benzentetrayl-tetrakis[pentanyl-(3-benzylpyridinium)]tetrabromide

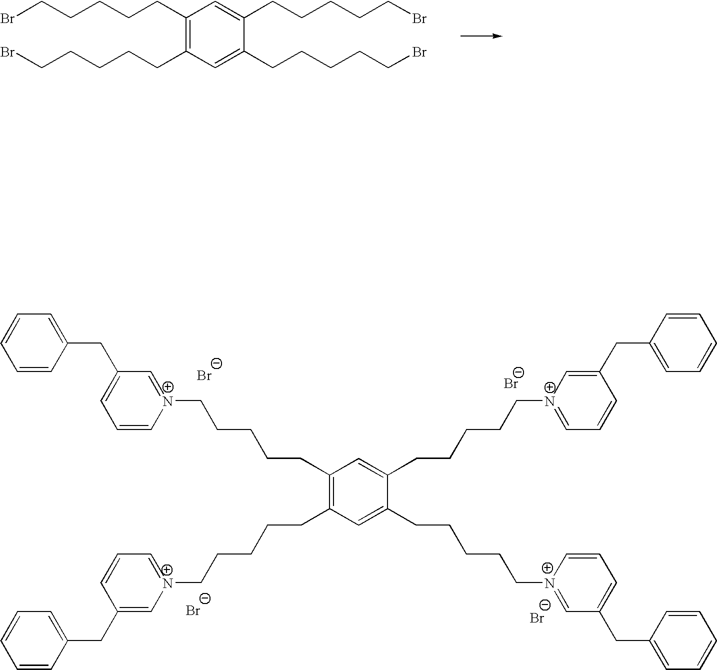

A mixture of 5,5′,5″,5′″-(1,2,4,5-benzentetrayl)tetrakis-[1-bromopentane] (330 mg, 0.49 mmol) and 3-benzyl-pyridine (390 mg, 2.3 mmol) was heated at 60-70° C. for 18 hrs. The resulting mixture was treated with diethyl ether and then dissolved in water (15 mL), the aqueous solution was extracted extensively with chloroform (30 mL×5). Water was removed through lyophilization to afford 575 mg of the title compound. Yield: 87%. ¹H NMR (300 MHz, CD₃OD) δ 9.20 (s, 4H, 8.97 (d, J=6.0 Hz, 4H), 8.41 (d, J=8.1 Hz, 4H), 8.00 (dd, 4H), 7.19-7.34 (m, 20H), 6.91 (s, 2H), 4.70 (t, J=7.5 Hz, 8H), 4.27 (s, 8H), 2.51-2.57 (m, 8H), 2.01-2.11 (m, 8H), 1.56-1.62 (m, 8H), 1.43-1.50 (m, 8H) ppm. ¹³C NMR, 145.61, 144.26, 143.36, 142.56, 138.18, 137.35, 130.18, 129.13, 129.06, 128.01, 127.17, 61.81, 38.03, 32.11, 31.56, 31.07, 26.23 ppm.

### Example 24

### Preparation of 5,5′,5″,5′″-(1,2,4,5-benzentetrayl)-tetrakis[pentanyl-(3-phenylpyridinium)]tetrabromide

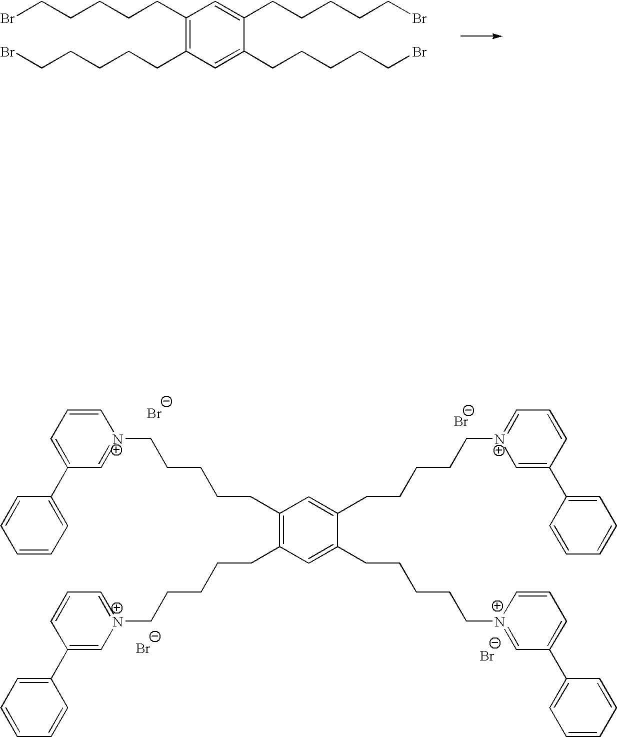

A mixture of 5,5′,5″,5′″-(1,2,4,5-benzentetrayl)tetrakis[1-bromopentane] (330 mg, 0.49 mmol) and 3-phenyl-pyridine (356 mg, 2.3 mmol) was heated at 60-70° C. for 18 hrs. The resulted mixture was treated with diethyl ether and then dissolved in water (15 mL), the aqueous solution was extracted extensively with chloroform (30 mL×5). Water was removed through lyophilization to afford 488 mg of the title compound. Yield: 77%. ¹H NMR (300 MHz, CD₃OD) δ 9.48 (s, 4H), 9.05 (d, J=6.0 Hz, 4H), 8.83 (d, J=8.4 Hz, 4H), 8.15 (dd, 4H), 7.87-7.90 (m, 8H), 7.50-7.88 (m, 12H), 6.85 (s, 2H), 4.80 (t, J=7.5 Hz, 8H), 2.48-2.54 (m, 8H), 2.08-2.14 (m, 8H), 1.45-1.62 (m, 16H) ppm. ¹³C NMR, 142.92, 142.78, 142.56, 141.18, 137.27, 133.26, 130.34, 130.13, 129.65, 128.36, 127.61, 62.05, 32.03, 31.70, 30.98, 26.20 ppm.

### Example 25

### Preparation of 5,5′,5″,5′″-(1,2,4,5-benzentetrayl)-tetrakis[pentanyl-(5,6,7,8-tetrahydroisoquinolinium)]tetrabromide

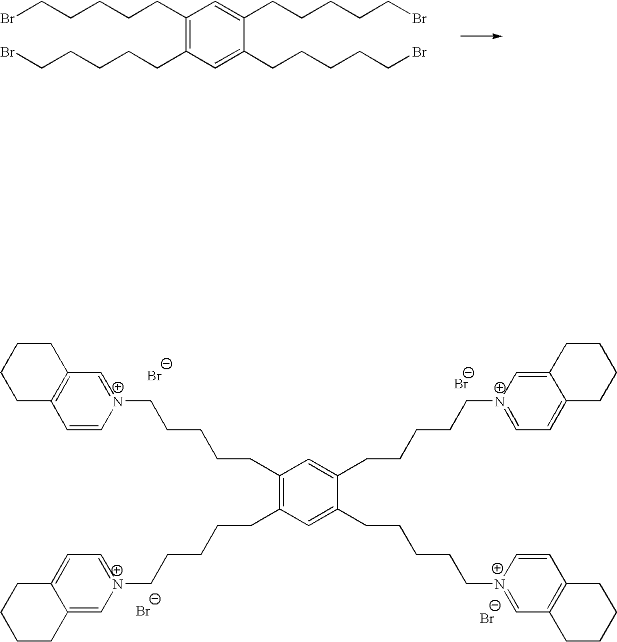

A mixture of 5,5′,5″,5′″-(1,2,4,5-benzentetrayl-tetrakis-[1-bromopentane] (330 mg, 0.49 mmol) and 5,6,7,8-tetrahydroisoqinoline (306 mg, 2.3 mmol) was heated at 60-70° C. for 18 hrs. The resulted mixture was treated with diethyl ether and then dissolved in water (15 mL), the aqueous solution was extracted extensively with chloroform (30 mL×5). Water was removed through lyophilization to afford 455 mg of the title compound. Yield: 77%. ¹H NMR (300 MHz, CD₃OD) δ 8.94 (s, 4H), 8.78 (d, J=6.3 Hz, 4H), 7.83 (d, J=6.3 Hz, 4H), 6.94 (s, 2H), 4.65 (t, J=7.5 Hz, 8H), 3.07 (br, 8H), 3.00 (br, 8H), 2.58 (m, 8H), 2.10 (m, 8H), 1.90 (br, 16H), 1.63 (br, 8H), 1.50 (br, 8H) ppm. ¹³C NMR, 158.33, 143.92, 140.37, 138.79, 137.41, 130.16, 128.02, 60.90, 32.17, 31.52, 31.21, 29.54, 26.41, 26.32, 21.37 ppm.

### Example 26

A rat model of tonic inflammatory pain (the formalin test) was used in this study (Wheeler-Aceto and Cowan, 1991). Fifty μl of formalin (5%) was injected subcutaneously (SC) into the dorsal surface of the left hind paw. This procedure typically produces a biphasic behavioral response consisting of flinching, lifting and licking. The first phase (0-10 minutes) results from direct stimulation of nociceptors (nociceptive pain) whereas the second phase (20-60 minutes) is thought to involve central sensitization. Rats (3/dose) were pretreated 15 minutes prior to formalin (SC) injection with ZZ-204G (3.6-3600 μg/kg) administered by the IP route. ZZ-204G is as below:

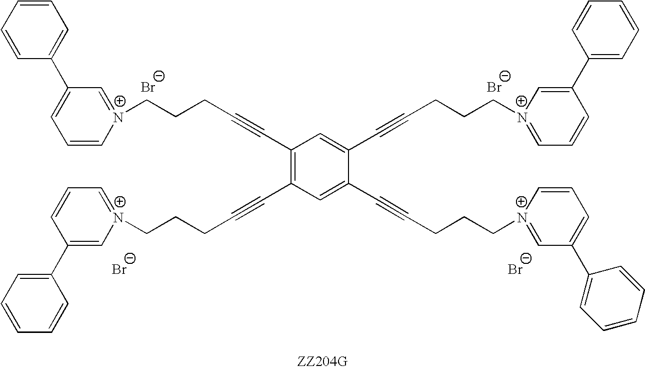

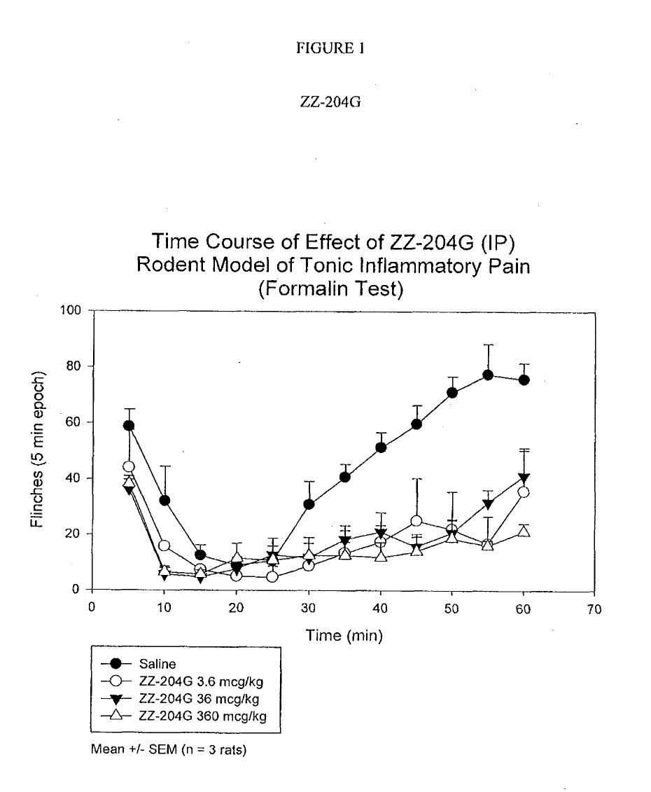

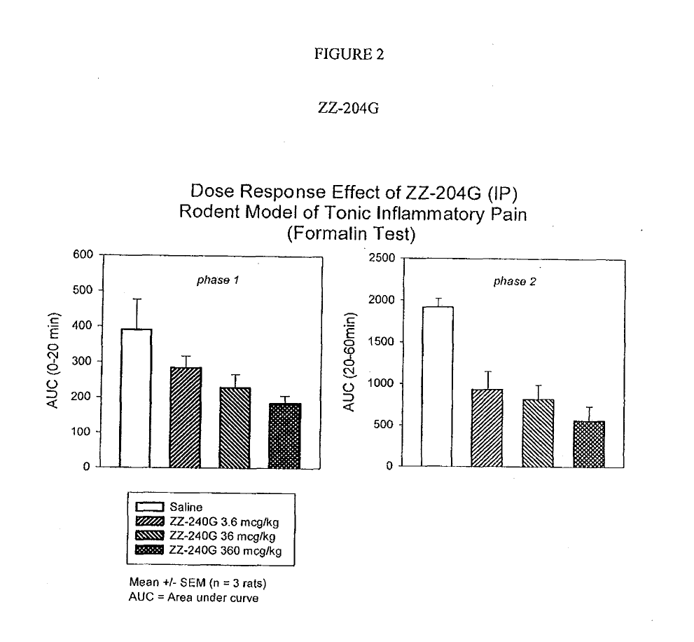

Saline served as control. Incidences of formalin-induced flinching were counted continuously in 5 minute intervals for 60 minutes. Each rat received only one treatment. The time course of the analgesic effect of ZZ-204G in the rodent formalin tonic inflammatory pain model following intraperitoneal administration was charted, as well as the dose response of the analgesic effect of ZZ-204G in phase 1 and 2 of the rodent formalin tonic inflammatory pain model following intraperitoneal administration. The results are further presented in FIGS. 1 and 2.

### Example 27

A rat model of neuropathic pain (the chronic constriction nerve injury model; CCI) was used. Unilateral peripheral mononeuropathy was produced on the left hind limb according to the method described by Bennett and Xie (1988). Under pentobarbital anesthesia (40 mg/kg, IP) ligation of the siatic nerve and sham surgery were performed in each rat on the left and right hind paws, respectively. Proximal to the sciatice trifurcation, the nerve (about 7 mm) was freed from adhering tissue and four loose ligatures were tied around the nerve (1 mm apart) using 4.0 chromic catgut, barely constricting the diameter of the nerve. In sham surgery, the right sciatic nerve was exposed using the same procedure, but the nerve was not ligated. The incision was closed in layers with silk thread 3.0. Rats showed a mild aversion of the affected paw and a mild degree of foot drop. No severe motor impairment was observed.

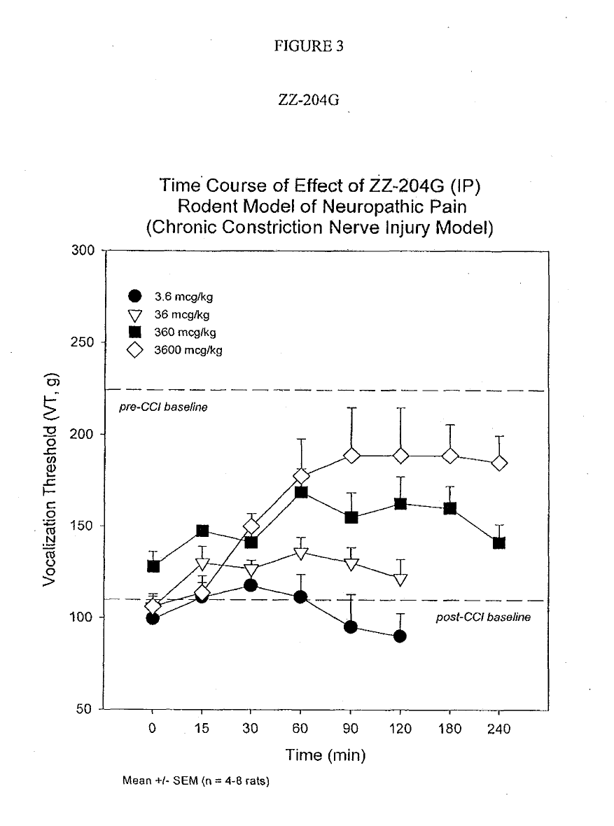

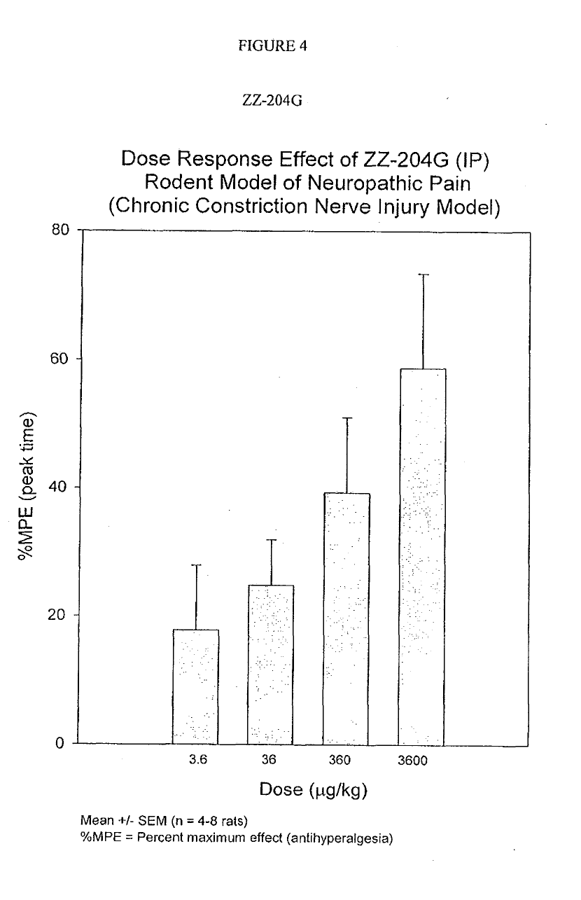

The presence of a decreased threshold to mechanical noxious stimuli (mechanical hyperalgesia) was evaluated by the Randal and Selitto (1957) method (paw pressure test) in CCI rats. The hind paw was placed between a flat surface and a blunt pointer in the Basile Analgesimeter (UGO Basile, Italy) and increasing pressure (32 g/s) was applied to the dorsal side of the paw. Vocalization was used as the end point (vocalization threshold, VT, g). Experiments were carried out at days 7, 9, 11, and 14 after surgery. At this time the abnormal pain behavior was at a stable maximum. The nerve-injured and sham-operated paws were tested alternatively in each rat. Responses were assessed prior to (baseline, taken twice) and at 15, 30, 45, 60 and 120 minutes after injection. Each rat received four doses (3.6-3600 μg/kg, IP) of ZZ-204G. Control rats were treated with saline. These results are further presented in FIGS. 3 and 4.

It will be appreciated that, although specific embodiments of the invention have been described herein for purposes of illustration, various modifications may be made without departing from the spirit and scope of the invention. All such modifications and variations are intended to be included herein within the scope of this disclosure and the present invention and protected by the following claims.

What is claimed is:

1. A method of modulating pain, comprising administering to a subject in need thereof a compound of Formula (I) 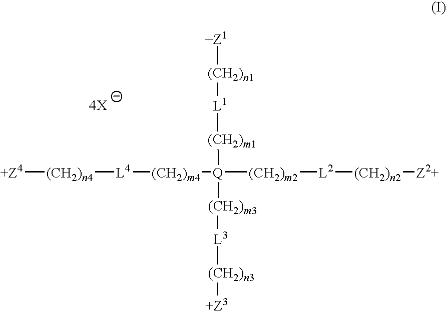
wherein each X^{θ} is independently an organic or inorganic anion;
wherein Q is a phenyl group substituted at the 1-, 2-, 3- and 4-positions, at the 1-, 2-, 3- and 5-positions, or at the 1-, 2-, 4- and 5-positions;
wherein m1, m2, m3 and m4 are each independently 0, 1, 2, 3, 4 or 5;
wherein n1, n2, n3 and n4 are each independently 1, 2, 3, 4 or 5;
wherein L¹, L², L³ and L⁴ are each independently selected from the group consisting of —CH₂CH₂—, cis —CH═CH—, trans —CH═CH—, —C≡C—, —S—CH₂—, —CH₂—S—, —Se—CH₂—, —CH₂—Se—, —O—CH₂—, —CH₂—O—, —NH—CH₂—, —CH₂—NH—, —N(lower alkyl)-CH₂—, —CH₂—N(lower alkyl)-, —N═CH—, —CH═N— and —N═N—;
wherein Z¹, Z², Z³ and Z⁴ are each independently five or six membered rings as shown in formulas (IIA) and (IIB), wherein each ring of Z¹, Z², Z³ and Z⁴ has one, two or three nitrogen atoms; 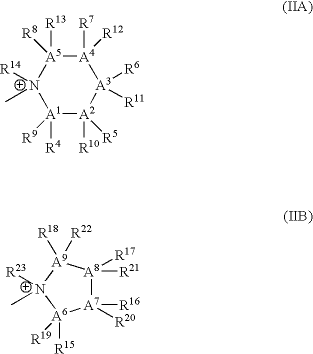
wherein A¹ is carbon or nitrogen, provided that when A¹ joins a ring atom with an unsaturated bond or is a nitrogen, R⁹ is absent, and when A¹ joins a ring atom with an unsaturated bond and is a nitrogen, both R⁴ and R⁹ are absent;
wherein A² is carbon or nitrogen, provided that when A² joins a ring atom with an unsaturated bond or is a nitrogen, R¹⁰ is absent, and when A² joins a ring atom with an unsaturated bond and is a nitrogen, both R⁵ and R¹⁰ are absent;
wherein A³ is carbon or nitrogen, provided that when A³ joins a ring atom with an unsaturated bond or is a nitrogen, R¹¹ is absent, and when A³ joins a ring atom with an unsaturated bond and is a nitrogen, both R⁶ and R¹¹ are absent;
wherein A⁴ is carbon or nitrogen, provided that when A⁴ joins a ring atom with an unsaturated bond or is a nitrogen, R¹² is absent, and when A⁴ joins a ring atom with an unsaturated bond and is a nitrogen, both R⁷ and R¹² are absent;
wherein A⁵ is carbon or nitrogen, provided that when A⁵ joins a ring atom with an unsaturated bond or is a nitrogen, R¹³ is absent, and when A⁵ joins a ring atom with an unsaturated bond and is a nitrogen, both R⁸ and R¹³ are absent;
wherein A⁶ is carbon or nitrogen, provided that when A⁶ joins a ring atom with an unsaturated bond or is a nitrogen, R¹⁹ is absent, and when A⁶ joins a ring atom with an unsaturated bond and is a nitrogen, both R¹⁵ and R¹⁹ are absent;
wherein A⁷ is carbon or nitrogen, provided that when A⁷ joins a ring atom with an unsaturated bond or is a nitrogen, R²⁰ is absent, and when A⁷ joins a ring atom with an unsaturated bond and is a nitrogen, both R¹⁶ and R²⁰ are absent;
wherein A⁸ is carbon or nitrogen, provided that when A⁸ joins a ring atom with an unsaturated bond or is a nitrogen, R²¹ is absent, and when A⁸ joins a ring atom with an unsaturated bond and is a nitrogen, both R¹⁷ and R²¹ are absent;
wherein A⁹ is carbon or nitrogen, provided that when A⁹ joins a ring atom with an unsaturated bond or is a nitrogen, R²² is absent, and when A⁹ joins a ring atom with an unsaturated bond and is a nitrogen, both R¹⁸ and R²² are absent;
wherein R¹⁴ or R²³ is absent when any of the bonds to the ammonium nitrogen is unsaturated, and R¹⁴ or R²³ is a straight chain or branched alkyl group of four carbons or fewer when all of the bonds to the ammonium nitrogen are saturated;
wherein R⁴, R⁵, R⁶, R⁷, R⁸, R⁹, R¹⁰, R¹¹, R¹², and R¹³ or R¹⁵, R¹⁶, R¹⁷, R¹⁸, R¹⁹, R²⁰, R²¹, and R²², when present, are each independently selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, substituted heterocyclic, halo, cyano, nitro, SOY¹, SO₂Y¹, SO₂OY¹ or SO₂NHY¹, where Y¹ is selected from hydrogen, lower alkyl, alkenyl, alkynyl or aryl, and where Y¹ is not hydrogen in SOY¹ and if Y¹ is alkenyl or alkynyl, the site of unsaturation is not conjugated with a heteroatom; COY², where Y² is selected from hydrogen, alkyl, substituted alkyl, cycloalkyl, substituted cycloalkyl, alkoxy, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, or substituted heterocyclic, and where if Y² comprises alkenyl or alkynyl, the site of unsaturation is not conjugated with the carbonyl group; OY³, where Y³ is selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, acyl, substituted acyl, alkylsulfonyl, arylsulfonyl, heterocyclic, or substituted heterocyclic, where if Y³ comprises alkenyl or alkynyl, the site of unsaturation is not conjugated with the oxygen; NY⁴Y⁵, where Y⁴ and Y⁵ are each independently selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, acyl, substituted acyl, alkylsulfonyl, arylsulfonyl, heterocyclic, or substituted heterocyclic, where if Y⁴ or Y⁵ comprises alkenyl or alkynyl, the site of unsaturation is not conjugated with the nitrogen; SY⁶, where Y⁶ is selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, or substituted heterocyclic, and where if Y⁶ comprises alkenyl or alkynyl, the site of unsaturation is not conjugated with the sulfur; or R⁴ and R⁵ together with A¹ and A², or R⁵ and R⁶ together with A² and A³, or R¹⁵ and R¹⁶ together with A⁶ and A⁷, or R¹⁶ and R¹⁷ together with A⁷ and A⁸ independently form a three to eight member cyclolkane, substituted cycloalkane, cycloalkene, substituted cycloalkene, aryl, substituted aryl, heterocycle with one to three hetero atoms in the ring, or substituted heterocycle with one to three hetero atoms in the ring.

2. The method of claim 1, wherein the phenyl ring of Q is 1,2,4,5-substituted;
wherein m1, m2, m3 and m4=0;
wherein n1, n2, n3 and n4=3;
wherein L¹, L², L³ and L⁴ are —CH₂CH₂— or —C≡C—;
wherein Z¹, Z², Z³ and Z⁴ are pyridinium rings;
wherein R⁴ is hydrogen;
wherein R⁵ is hydrogen, methyl, hydroxypropyl, phenyl, benzyl, 1-methyl-2-pyrrolidinyl, forms a phenyl group with A², A³ and R⁶, or forms a cyclohexyl group with A², A³ and R⁶;
wherein R⁶ is hydrogen, methyl, forms a phenyl group with A², A³ and R⁵, or forms a cyclohexyl group with A², A³ and R⁵; and
wherein X^{θ} is Br.

3. The method of claim 1, wherein the phenyl ring of Q is 1,2,4,5-substituted;
wherein m1, m2, m3 and m4=0;
wherein n1, n2, n3 and n4=3;
wherein L¹, L², L³ and L⁴ are —CH₂CH₂—;
wherein Z¹, Z², Z³ and Z⁴ are pyridinium rings;
wherein R⁴ is hydrogen;
wherein R⁵ is hydrogen, methyl, hydroxypropyl, phenyl, benzyl, 1-methyl-2-pyrrolidinyl, forms a phenyl group with A², A³ and R⁶, or forms a cyclohexyl group with A², A³ and R⁶;
wherein R⁶ is hydrogen, methyl, forms a phenyl group with A², A³ and R⁵, or forms a cyclohexyl group with A², A³ and R⁵; and
wherein X^{θ} is Br.

4. The method of claim 1, wherein the phenyl ring of Q is 1,2,4,5-substituted;
wherein m1, m2, m3 and m4=0;
wherein n1, n2, n3 and n4=3;
wherein L¹, L², L³ and L⁴ are —C≡C—;
wherein Z¹, Z², Z³ and Z⁴ are pyridinium rings;
wherein R⁴ is hydrogen;
wherein R⁵ is hydrogen, methyl, hydroxypropyl, phenyl, benzyl, 1-methyl-2-pyrrolidinyl, forms a phenyl group with A², A³ and R⁶, or forms a cyclohexyl group with A², A³ and R⁶;
wherein R⁶ is hydrogen, methyl, forms a phenyl group with A², A³ and R⁵, or forms a cyclohexyl group with A², A³ and R⁵; and
wherein X^{θ} is Br.

5. The method of claim 1 selected from the group consisting of:
5,5′,5″,5′″-(1,2,4,5-benzentetrayl)-tetrakis[4-pentyn-1-yl-(3-methylpyridinium)]tetrabromide;
5,5′,5″,5′″-(1,2,4,5-benzentetrayl)-tetrakis[4-pentyn-1-yl-(4-methylpyridinium)]tetrabromide;
5,5′,5″,5′″-(1,2,4,5-benzentetrayl)-tetrakis[4-pentyn-1-yl-(3,4-dimethylpyridinium)]tetrabromide;
5,5′,5″,5′″-(1,2,4,5-benzentetrayl)-tetrakis[4-pentyn-1-yl-(3,5-dimethylpyridinium)]tetrabromide;
5,5′,5″,5′″-(1,2,4,5-benzentetrayl)-tetrakis[4-pentyn-1-yl-nicotinium) tetrabromide;
5,5′,5″,5′″-(1,2,4,5-benzentetrayl)tetrakis[4-pentyn-1-yl-(5,6,7,8-tetrahydroisoquinolinium)]tetrabromide;
5,5′,5″,5′″-(1,2,4,5-benzentetrayl)-tetrakis[4-pentyn-1-yl-(3-phenyl-pyridinium)]tetrabromide;
5,5′,5″,5′″-(1,2,4,5-benzentetrayl)-tetrakis(4-pentyn-1-yl-isoquinolinolinium) tetrabromide;
5,5′,5″,5′″-(1,2,4,5-benzentetrayl)-tetrakis[4-pentyn-1-yl-(3-benzyl-pyridinium)]tetrabromide;
5,5′,5″,5′″-(1,2,4,5-benzentetrayl)-tetrakis {4-pentyn-1-yl-[3-(3-hydroxypropyl)-pyridinium]}tetrabromide;
5,5′,5″,5′″-(1,2,4,5-benzentetrayl)-tetrakis[pentanyl-(3-methylpyridinium)]tetrabromide;
5,5′,5″,5′″-(1,2,4,5-benzentetrayl)-tetrakis[pentanyl-(4-methylpyridinium)]tetrabromide;
5,5′,5″,5′″-(1,2,4,5-benzentetrayl)-tetrakis[pentanyl-(3,4-dimethylpyridinium)]tetrabromide;
5,5′,5″,5′″-(1,2,4,5-benzentetrayl)-tetrakis[pentanyl-(3,5-dimethylpyridinium)]tetrabromide;
5,5′,5″,5′″-(1,2,4,5-benzentetrayl)-tetrakis[pentanyl-(nicotinium)]tetrabromide;
5,5′,5″,5′″-(1,2,4,5-benzentetrayl)-tetrakis[pentanyl-(3-(3-hydroxypropanyl)pyridinium)]tetrabromide;
5,5′,5″,5′″-(1,2,4,5-benzentetrayl)-tetrakis[pentanyl-(isoquinolinium)]tetrabromide;
5,5′,5″,5′″-(1,2,4,5-benzentetrayl)-tetrakis[pentanyl-(3-benzylpyridinium)]tetrabromide;
5,5′,5″,5′″-(1,2,4,5-benzentetrayl)-tetrakis[pentanyl-(3-phenylpyridinium)]tetrabromide; and
5,5′,5″,5′″-(1,2,4,5-benzentetrayl)-tetrakis[pentanyl-(5,6,7,8-tetrahydroisoquinolinium)]tetrabromide.

6. A method of modulating pain comprising administering to a subject in need thereof a composition comprising a pharmaceutically acceptable carrier and a compound of claim 1.

7. A method of modulating pain comprising administering to a subject in need thereof a composition comprising a pharmaceutically acceptable carrier and a compound of claim 2.

8. A method of modulating pain comprising administering to a subject in need thereof a composition comprising a pharmaceutically acceptable carrier and a compound of claim 3.

9. A method of modulating pain comprising administering to a subject in need thereof a composition comprising a pharmaceutically acceptable carrier and a compound of claim 4.

10. A method of modulating pain comprising administering to a subject in need thereof a composition comprising a pharmaceutically acceptable carrier and a compound of claim 5.

11. A method of modulating pain comprising administering to a subject in need thereof a therapeutically effective amount of a compound of Formula (I): 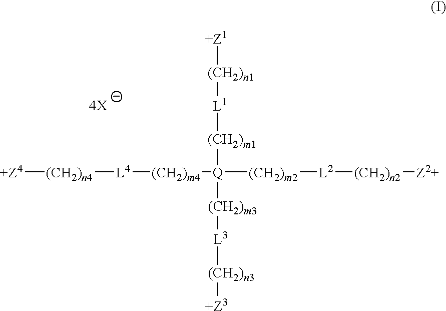
wherein each X^{θ} is independently an organic or inorganic anion;
wherein Q is a phenyl group substituted at the 1-, 2-, 3- and 4-positions, at the 1-, 2-, 3- and 5-positions, or at the 1-, 2-, 4- and 5-positions;
wherein m1, m2, m3 and m4 are each independently 0, 1, 2, 3, 4 or 5;
wherein n1, n2, n3 and n4 are each independently 1, 2, 3, 4 or 5;
wherein L¹, L², L³ and L⁴ are each independently selected from the group consisting of —CH₂CH₂—, cis —CH═CH—, trans —CH═CH—, —C≡C—, —S—CH₂—, —CH₂—S—, —Se—CH₂—, —CH₂—Se—, —O—CH₂—, —CH₂—O—, —NH—CH₂—, —CH₂—NH—, —N(lower alkyl)-CH₂—, —CH₂—N(lower alkyl)-, —N═CH—, —CH═N— and —N═N—;
wherein Z¹, Z², Z³ and Z⁴ are each independently five or six membered rings as shown in formulas (IIA) and (IIB), wherein each ring of Z¹, Z², Z³ and Z⁴ has one, two or three nitrogen atoms; 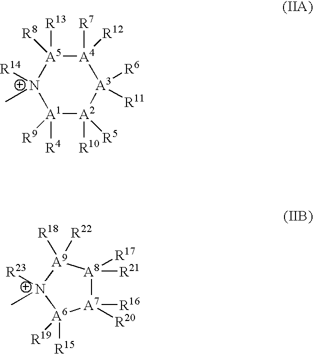
wherein A¹ is carbon or nitrogen, provided that when A¹ joins a ring atom with an unsaturated bond or is a nitrogen, R⁹ is absent, and when A¹ joins a ring atom with an unsaturated bond and is a nitrogen, both R⁴ and R⁹ are absent;
wherein A² is carbon or nitrogen, provided that when A² joins a ring atom with an unsaturated bond or is a nitrogen, R¹⁰ is absent, and when A² joins a ring atom with an unsaturated bond and is a nitrogen, both R⁵ and R¹⁰ are absent;
wherein A³ is carbon or nitrogen, provided that when A³ joins a ring atom with an unsaturated bond or is a nitrogen, R¹¹ is absent, and when A³ joins a ring atom with an unsaturated bond and is a nitrogen, both R⁶ and R¹¹ are absent;
wherein A⁴ is carbon or nitrogen, provided that when A⁴ joins a ring atom with an unsaturated bond or is a nitrogen, R¹² is absent, and when A⁴ joins a ring atom with an unsaturated bond and is a nitrogen, both R⁷ and R¹² are absent;
wherein A⁵ is carbon or nitrogen, provided that when A⁵ joins a ring atom with an unsaturated bond or is a nitrogen, R¹³ is absent, and when A⁵ joins a ring atom with an unsaturated bond and is a nitrogen, both R⁸ and R¹³ are absent;
wherein A⁶ is carbon or nitrogen, provided that when A⁶ joins a ring atom with an unsaturated bond or is a nitrogen, R¹⁹ is absent, and when A⁶ joins a ring atom with an unsaturated bond and is a nitrogen, both R¹⁵ and R¹⁹ are absent;
wherein A⁷ is carbon or nitrogen, provided that when A⁷ joins a ring atom with an unsaturated bond or is a nitrogen, R²⁰ is absent, and when A⁷ joins a ring atom with an unsaturated bond and is a nitrogen, both R¹⁶ and R²⁰ are absent;
wherein A⁸ is carbon or nitrogen, provided that when A⁸ joins a ring atom with an unsaturated bond or is a nitrogen, R²¹ is absent, and when A⁸ joins a ring atom with an unsaturated bond and is a nitrogen, both R¹⁷ and R²¹ are absent;
wherein A⁹ is carbon or nitrogen, provided that when A⁹ joins a ring atom with an unsaturated bond or is a nitrogen, R²² is absent, and when A⁹ joins a ring atom with an unsaturated bond and is a nitrogen, both R¹⁸ and R²² are absent;
wherein R¹⁴ or R²³ is absent when any of the bonds to the ammonium nitrogen is unsaturated, and R¹⁴ or R²³ is a straight chain or branched alkyl group of four carbons or fewer when all of the bonds to the ammonium nitrogen are saturated;
wherein R⁴, R⁵, R⁶, R⁷, R⁸, R⁹, R¹⁰, R¹¹, R¹², and R¹³ or R¹⁵, R¹⁶, R¹⁷, R¹⁸, R¹⁹, R²⁰, R²¹, and R²², when present, are each independently selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, substituted heterocyclic, halo, cyano, nitro, SOY¹, SO₂Y¹, SO₂OY¹ or SO₂NHY¹, where Y¹ is selected from hydrogen, lower alkyl, alkenyl, alkynyl or aryl, and where Y¹ is not hydrogen in SOY¹ and if Y¹ is alkenyl or alkynyl, the site of unsaturation is not conjugated with a heteroatom; COY², where Y² is selected from hydrogen, alkyl, substituted alkyl, cycloalkyl, substituted cycloalkyl, alkoxy, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, or substituted heterocyclic, and where if Y² comprises alkenyl or alkynyl, the site of unsaturation is not conjugated with the carbonyl group; OY³, where Y³ is selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, acyl, substituted acyl, alkylsulfonyl, arylsulfonyl, heterocyclic, or substituted heterocyclic, where if Y³ comprises alkenyl or alkynyl, the site of unsaturation is not conjugated with the oxygen; NY⁴Y⁵, where Y⁴ and Y⁵ are each independently selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, acyl, substituted acyl, alkylsulfonyl, arylsulfonyl, heterocyclic, or substituted heterocyclic, where if Y⁴ or Y⁵ comprises alkenyl or alkynyl, the site of unsaturation is not conjugated with the nitrogen; SY⁶, where Y⁶ is selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, or substituted heterocyclic, and where if Y⁶ comprises alkenyl or alkynyl, the site of unsaturation is not conjugated with the sulfur; or R⁴ and R⁵ together with A¹ and A², or R⁵ and R⁶ together with A² and A³, or R¹⁵ and R¹⁶ together with A⁶ and A⁷, or R¹⁶ and R¹⁷ together with A⁷ and A⁸ independently form a three to eight member cyclolkane, substituted cycloalkane, cycloalkene, substituted cycloalkene, aryl, substituted aryl, heterocycle with one to three hetero atoms in the ring, or substituted heterocycle with one to three hetero atoms in the ring.

12. The method of claim 11, wherein the compound of Formula (I) is selected from the group consisting of:
5,5′,5″,5′″-(1,2,4,5-benzentetrayl)-tetrakis[4-pentyn-1-yl-(3-methylpyridinium)]tetrabromide;
5,5′,5″,5′″-(1,2,4,5-benzentetrayl)-tetrakis[4-pentyn-1-yl-(4-methylpyridinium)]tetrabromide;
5,5′,5″,5′″-(1,2,4,5-benzentetrayl)-tetrakis[4-pentyn-1-yl-(3,4-dimethylpyridinium)]tetrabromide;
5,5′,5″,5′″-(1,2,4,5-benzentetrayl)-tetrakis[4-pentyn-1-yl-(3,5-dimethylpyridinium)]tetrabromide;
5,5′,5″,5′″-(1,2,4,5-benzentetrayl)-tetrakis[4-pentyn-1-yl-nicotinium) tetrabromide;
5,5′,5″,5′″-(1,2,4,5-benzentetrayl)tetrakis[4-pentyn-1-yl-(5,6,7,8-tetrahydroisoquinolinium)]tetrabromide;
5,5′,5″,5′″-(1,2,4,5-benzentetrayl)-tetrakis[4-pentyn-1-yl-(3-phenyl-pyridinium)]tetrabromide;
5,5′,5″,5′″-(1,2,4,5-benzentetrayl)-tetrakis(4-pentyn-1-yl-isoquinolinolinium) tetrabromide;
5,5′,5″,5′″-(1,2,4,5-benzentetrayl)-tetrakis[4-pentyn-1-yl-(3-benzyl-pyridinium)]tetrabromide;
5,5′,5″,5′″-(1,2,4,5-benzentetrayl)-tetrakis {4-pentyn-1-yl-[3-(3-hydroxypropyl)-pyridinium]}tetrabromide;
5,5′,5″,5′″-(1,2,4,5-benzentetrayl)-tetrakis[pentanyl-(3-methylpyridinium)]tetrabromide; 5,5′,5″,5′″-(1,2,4,5-benzentetrayl)-tetrakis[pentanyl-(4-methylpyridinium)]tetrabromide;
5,5′,5″,5′″-(1,2,4,5-benzentetrayl)-tetrakis[pentanyl-(3,4-dimethylpyridinium)]tetrabromide;
5,5′,5″,5′″-(1,2,4,5-benzentetrayl)-tetrakis[pentanyl-(3,5-dimethylpyridinium)]tetrabromide;
5,5′,5″,5′″-(1,2,4,5-benzentetrayl)-tetrakis[pentanyl-(nicotinium)]tetrabromide;
5,5′,5″,5′″-(1,2,4,5-benzentetrayl)-tetrakis[pentanyl-(3-(3-hydroxypropanyl)pyridinium)]tetrabromide;
5,5′,5″,5′″-(1,2,4,5-benzentetrayl)-tetrakis[pentanyl-(isoquinolinium)]tetrabromide;
5,5′,5″,5′″-(1,2,4,5-benzentetrayl)-tetrakis[pentanyl-(3-benzylpyridinium)]tetrabromide;
5,5′,5″,5′″-(1,2,4,5-benzentetrayl)-tetrakis[pentanyl-(3-phenylpyridinium)]tetrabromide; and
5,5′,5″,5′″-(1,2,4,5-benzentetrayl)-tetrakis[pentanyl-(5,6,7,8-tetrahydroisoquinolinium)]tetrabromide.

13. A method for selectively modulating pain comprising administering to a mammalian subject in need thereof a therapeutically effective amount of a compound of Formula (I): 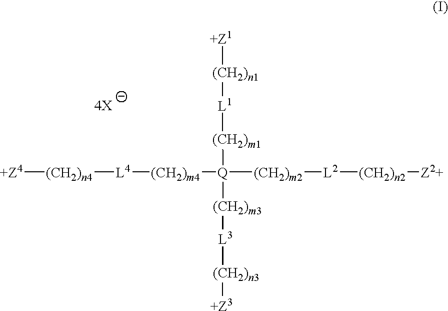
wherein each X^{θ} is independently an organic or inorganic anion;
wherein Q is a phenyl group substituted at the 1-, 2-, 3- and 4-positions, at the 1-, 2-, 3- and 5-positions, or at the 1-, 2-, 4- and 5-positions;
wherein m1, m2, m3 and m4 are each independently 0, 1, 2, 3, 4 or 5;
wherein n1, n2, n3 and n4 are each independently 1, 2, 3, 4 or 5;
wherein L¹, L², L³ and L⁴ are each independently selected from the group consisting of —CH₂CH₂—, cis —CH═CH—, trans —CH═CH—, —C≡C—, —S—CH₂, —CH₂—S—, —Se—CH₂, —CH₂—Se—, —O—CH₂—, —CH₂—O—, —NH—CH₂, —CH₂—NH—, —N(lower alkyl)-CH₂—, —CH₂—N(lower alkyl)-, —N═CH—, —CH═N— and —N═N—;
wherein Z¹, Z², Z³ and Z⁴ are each independently five or six membered rings as shown in formulas (IIA) and (IIB), wherein each ring of Z¹, Z², Z³ and Z⁴ has one, two or three nitrogen atoms; 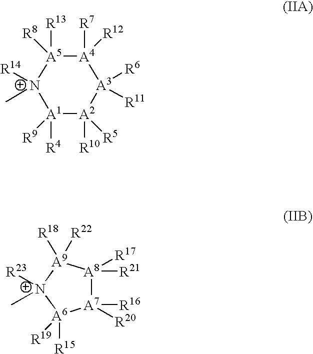
wherein A¹ is carbon or nitrogen, provided that when A¹ joins a ring atom with an unsaturated bond or is a nitrogen, R⁹ is absent, and when A¹ joins a ring atom with an unsaturated bond and is a nitrogen, both R⁴ and R⁹ are absent;
wherein A² is carbon or nitrogen, provided that when A² joins a ring atom with an unsaturated bond or is a nitrogen, R¹⁰ is absent, and when A² joins a ring atom with an unsaturated bond and is a nitrogen, both R⁵ and R¹⁰ are absent;
wherein A³ is carbon or nitrogen, provided that when A³ joins a ring atom with an unsaturated bond or is a nitrogen, R¹¹ is absent, and when A³ joins a ring atom with an unsaturated bond and is a nitrogen, both R⁶ and R¹¹ are absent;
wherein A⁴ is carbon or nitrogen, provided that when A⁴ joins a ring atom with an unsaturated bond or is a nitrogen, R¹² is absent, and when A⁴ joins a ring atom with an unsaturated bond and is a nitrogen, both R⁷ and R¹² are absent;
wherein A⁵ is carbon or nitrogen, provided that when A⁵ joins a ring atom with an unsaturated bond or is a nitrogen, R¹³ is absent, and when A⁵ joins a ring atom with an unsaturated bond and is a nitrogen, both R⁸ and R¹³ are absent;
wherein A⁶ is carbon or nitrogen, provided that when A⁶ joins a ring atom with an unsaturated bond or is a nitrogen, R¹⁹ is absent, and when A⁶ joins a ring atom with an unsaturated bond and is a nitrogen, both R¹⁵ and R¹⁹ are absent;
wherein A⁷ is carbon or nitrogen, provided that when A⁷ joins a ring atom with an unsaturated bond or is a nitrogen, R²⁰ is absent, and when A⁷ joins a ring atom with an unsaturated bond and is a nitrogen, both R¹⁶ and R²⁰ are absent;
wherein A⁸ is carbon or nitrogen, provided that when A⁸ joins a ring atom with an unsaturated bond or is a nitrogen, R²¹ is absent, and when A⁸ joins a ring atom with an unsaturated bond and is a nitrogen, both R¹⁷ and R²¹ are absent;
wherein A⁹ is carbon or nitrogen, provided that when A⁹ joins a ring atom with an unsaturated bond or is a nitrogen, R²² is absent, and when A⁹ joins a ring atom with an unsaturated bond and is a nitrogen, both R¹⁸ and R²² are absent;
wherein R¹⁴ or R²³ is absent when any of the bonds to the ammonium nitrogen is unsaturated, and R¹⁴ or R²³ is a straight chain or branched alkyl group of four carbons or fewer when all of the bonds to the ammonium nitrogen are saturated;
wherein R⁴, R⁵, R⁶, R⁷, R⁸, R⁹, R¹⁰, R¹¹, R¹² and R¹³ or R¹⁵, R¹⁶, R¹⁷, R¹⁸, R¹⁹, R²⁰, R²¹, and R²², when present, are each independently selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, substituted heterocyclic, halo, cyano, nitro, SOY¹, SO₂Y¹, SO₂OY¹ or SO₂NHY¹, where Y¹ is selected from hydrogen, lower alkyl, alkenyl, alkynyl or aryl, and where Y¹ is not hydrogen in SOY¹ and if Y¹ is alkenyl or alkynyl, the site of unsaturation is not conjugated with a heteroatom; COY², where Y² is selected from hydrogen, alkyl, substituted alkyl, cycloalkyl, substituted cycloalkyl, alkoxy, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, or substituted heterocyclic, and where if Y² comprises alkenyl or alkynyl, the site of unsaturation is not conjugated with the carbonyl group; OY³, where Y³ is selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, acyl, substituted acyl, alkylsulfonyl, arylsulfonyl, heterocyclic, or substituted heterocyclic, where if Y³ comprises alkenyl or alkynyl, the site of unsaturation is not conjugated with the oxygen; NY⁴Y⁵, where Y⁴ and Y⁵ are each independently selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, acyl, substituted acyl, alkylsulfonyl, arylsulfonyl, heterocyclic, or substituted heterocyclic, where if Y⁴ or Y⁵ comprises alkenyl or alkynyl, the site of unsaturation is not conjugated with the nitrogen; SY⁶, where Y⁶ is selected from hydrogen, alkyl, substituted alkyl, cycloalkyl, substituted cycloalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, or substituted heterocyclic, and where if Y⁶ comprises alkenyl or alkynyl, the site of unsaturation is not conjugated with the sulfur; or R⁴ and R⁵ together with A¹ and A², or R⁵ and R⁶ together with A² and A³, or R¹⁵ and R¹⁶ together with A⁶ and A⁷, or R¹⁶ and R¹⁷ together with A⁷ and A⁸ independently form a three to eight member cyclolkane, substituted cycloalkane, cycloalkene, substituted cycloalkene, aryl, substituted aryl, heterocycle with one to three hetero atoms in the ring, or substituted heterocycle with one to three hetero atoms in the ring.

14. The method of claim 1, wherein the pain is inflammatory pain.

15. The method of claim 14, wherein the inflammatory pain results from a condition selected from the group consisting of spondylitis, benign prostatic hyperplasia, cholecystitis, ulcerative colitis, Crohn's disease, diabetes mellitus, gastritis, glomerulonephritis, irritable bowel syndrome, multiple sclerosis, osteoarthritis, pancreatitis, polymyositis, psoriasis and rheumatoid arthritis.
